(19) United States
(12) Patent Application Publication
(10) Pub. No.: US 2020/0306325 A1
(43) Pub. Date: Oct. 1, 2020

(54) PROBIOTIC CONSORTIA FOR ORAL HEALTH

(71) Applicant: Oralta, Inc., San Francisco, CA (US)

(72) Inventors: Patrick SCHWIENTEK, Davis, CA (US); Kishore NANNAPANENI, Davis, CA (US); David DRAKE, Iowa City, IA (US); Jeffrey Alan BANAS, Solon, IA (US)

(21) Appl. No.: 16/811,517

(22) Filed: Mar. 6, 2020

Related U.S. Application Data
(60) Provisional application No. 62/972,542, filed on Feb. 10, 2020.
(60) Provisional application No. 62/814,399, filed on Mar. 6, 2019.

(51) Int. Cl. A61K 35/747; A61K 35/745; A61K 35/744; A61K 33/30; A61K 9/00; A61K 47/46; A61K 47/10; A61P 31/04

(52) U.S. Cl. CPC A61K 35/747; A61K 35/745; A61K 35/744; A61P 31/04; A61K 9/0056; A61K 47/46; A61K 47/10; A61K 33/30

(57) ABSTRACT

The present disclosure provides methods and composition for preventing and/or treating oral diseases or conditions (e.g., halitosis, a periodontal disease, or caries) in a subject (e.g., a human) in need thereof. The compositions described herein may allow for oral administration, e.g., using a tablet dosage form. The compositions may comprise one or more probiotics.

### CROSS-REFERENCE

This application claims the benefit of U.S. Provisional Application Nos. 62/972,542, filed Feb. 10, 2020, and 62/814,399, filed Mar. 6, 2019, which applications are incorporated herein by reference in their entireties.

### BACKGROUND

Oral conditions such as halitosis, a periodontal disease, or caries may be caused by a dysbiosis of microorganisms inhabiting the oral cavity. One or more bacterial species that are naturally occurring even in healthy individuals can become overly abundant to a point where those microorganisms reach pathogenic levels either in the entirety of the oral cavity or in isolated spaces, such as lesions or pockets between gum and tooth. To date, chemical or other invasive treatments such as mouth washes or dental procedures are used to treat or prevent oral diseases such as gum disease or caries that may be caused by such bacteria. However, such methods are often invasive, have poor patient compliance, and do not discriminate between detrimental bacteria and beneficial bacteria and thus can result in even more severe disease states over time.

Mouth washes and other chemicals intended to treat oral diseases (e.g., halitosis, periodontal disease, and/or caries) may indiscriminately kill all bacteria present in an oral microbiome (e.g., including bacteria that may form a healthy oral microbiome). One of the disadvantages of these conventional approaches is that their beneficial and/or therapeutic effect may only last for short periods of time. Moreover, conventional methods to treat oral diseases such as halitosis, periodontal disease, and/or caries may not hinder the regrowth of detrimental bacteria. Due to a likely overabundance of detrimental bacteria in the first place, it is likely that more detrimental bacteria may survive the mouthwash treatment compared to non-detrimental (e.g., beneficial) bacteria. This effect may be further amplified as detrimental bacteria may be more likely to survive in protected sites, which may often correlate with sites of disease (e.g., periodontal pockets, cavities, or crevices on the tongue dorsum). This may cause an even greater overabundance of detrimental bacteria relative to beneficial bacteria.

Thus, there exists an unmet need to help the millions of people who suffer from oral conditions by providing alternative and superior prevention and/or treatment options for oral diseases that address the root causes of such diseases rather than merely treating their symptoms.

### SUMMARY

The present disclosure provides methods and compositions for preventing and/or treating oral diseases or conditions (e.g., halitosis, periodontal disease, caries, etc.) and/or systemic diseases that can be caused by or be otherwise influenced by the oral microbiome of a subject (e.g., a human). The compositions described herein may allow for oral administration, e.g., by using an oral dosage form such as a tablet or a capsule. Such compositions can comprise one or more probiotic microorganisms (e.g., beneficial microbial species) such as a bacterial population comprising one or more different bacterial species. Such a bacterial population, either alone and/or in combination with one or more additives or excipients, can inhibit the growth of pathogenic microorganisms in an oral cavity of a subject.

In various aspects, provided herein is a pharmaceutical composition for treating an oral condition in a subject, said composition comprising: (i) at least one probiotic microorganism; (ii) at least one VSC-releasing enzyme inhibitor; and (iii) at least one sweetener; wherein said composition comprises at least 10⁹ CFU of said at least one probiotic microorganism. In some aspects, said at least one probiotic microorganism comprises at least one Lactobacillus sp., at least one Lactococcus sp., at least one Bifidobacterium sp., at least one Streptococcus sp., or a combination thereof. In some aspects, said at least one Lactobacillus sp. is Lactobacillus reuteri, Lactobacillus salivarius, Lactobacillus brevis, Lactobacillus rhamnosus, Lactobacillus acidophilus, Lactobacillus plantarum, Lactobacillus casei, Lactobacillus paracasei, Lactobacillus curvatus, or a combination thereof. In some aspects, said at least one Lactococcus sp. is Lactococcus lactis. In some aspects, said at least one Bifidobacterium sp. is Bifidobacterium longum. In some aspects, said at least one Streptococcus sp. is Streptococcus salivarius. In some aspects, said Streptococcus salivarius strain is Streptococcus salivarius K12, Streptococcus salivarius M18, or a combination thereof. In some aspects, said at least one VSC-releasing enzyme inhibitor comprises zinc. In some aspects, said at least one VSC-releasing enzyme inhibitor is zinc oxide. In some aspects, said at least one sweetener is a stevia leaf extract, xylitol, a derivative thereof, or a combination thereof. In some aspects, said pharmaceutical composition comprises at least 5×10⁹ CFU to 10¹¹ CFU of said at least one probiotic microorganism. In some aspects, said pharmaceutical composition further comprises at least one filler. In some aspects, said at least one filler is microcrystalline cellulose, stearic acid, magnesium stearate, silicon dioxide, a derivative thereof, or a combination thereof. In some aspects, said pharmaceutical composition further comprises at least one prebiotic. In some aspects, said at least one prebiotic is maltodextrin or a derivative thereof. In some aspects, said pharmaceutical composition further comprises at least one flavoring. In some aspects, said at least one flavoring is a natural mint flavor. In some aspects, said pharmaceutical composition further comprises at least one BLIS fermentation media remnant. In some aspects, said at least one BLIS fermentation media remnant is trehalose, lactitol, a derivative thereof, or a combination thereof. In some aspects, said pharmaceutical composition further comprises at least one vitamin. In some aspects, said at least one vitamin is vitamin C, a vitamin D, a vitamin B, or any combination thereof. In some aspects, said pharmaceutical composition further comprises at least one mineral. In some aspects, said at least one mineral is iron, calcium, magnesium, or a combination thereof. In some aspects, said pharmaceutical composition is formulated in an oral dosage form. In some aspects, said oral dosage form is a capsule or a tablet. In some aspects, said oral dosage form comprises a coating. In some aspects, said coating comprises at least one substance that enhances a taste, texture, nutritional value, efficacy, or combination thereof of said oral dosage form. In some aspects, said coating comprises a prebiotic. In some aspects, said oral dosage form disintegrates in an oral cavity of a subject. In some aspects, said oral dosage form disintegrates in said oral cavity from 1 minute to 10 minutes after administration of said oral dosage form to said subject. In some aspects, said oral dosage form disintegrates in said oral cavity from 2 minutes to 5 minutes after administration of said oral dosage form to said subject.

In various aspects, provided herein is a method for treating an oral condition in a subject in need thereof, said method comprising: (i) administering a therapeutically effective amount of a pharmaceutical composition comprising at least one probiotic microorganism; (ii) increasing one or more of the following in an oral cavity of the subject: (a) a number of beneficial bacteria by at least one order of magnitude; and/or (b) a saliva pH to at least about 6.5 but no more than about 7.5; and (iii) decreasing a number of pathogenic bacteria by at least one order of magnitude in the oral cavity of the subject.

In various aspects, provided herein is a method for treating a systemic condition in a subject that is linked to or has been caused by an oral condition in said subject, said method comprising: (i) administering a therapeutically effective amount of a pharmaceutical composition comprising at least one probiotic microorganism; and (ii) inhibiting one or more systemic disease pathways linked to said oral condition. In some aspects, said oral condition is associated with or caused by an oral dysbiosis. In some aspects, said oral condition is halitosis, a periodontal disease, caries, or a combination thereof. In some aspects, said systemic condition is cardiovascular disease, arthritis, obesity, diabetes, bacterial pneumonia, or preterm birth. In some aspects, said at least one probiotic microorganism comprises at least one Lactobacillus sp., at least one Lactococcus sp., at least one Bifidobacterium sp., at least one Streptococcus sp., or a combination thereof. In some aspects, said at least one Lactobacillus sp. is Lactobacillus reuteri, Lactobacillus salivarius, Lactobacillus brevis, Lactobacillus rhamnosus, Lactobacillus acidophilus, Lactobacillus plantarum, or a combination thereof. In some aspects, said at least one Lactococcus sp. is Lactococcus lactis. In some aspects, said at least one Bifidobacterium sp. is Bifidobacterium longum. In some aspects, said at least one Streptococcus sp. is Streptococcus salivarius. In some aspects, said Streptococcus salivarius strain is Streptococcus salivarius K12, Streptococcus salivarius M18, or a combination thereof. In some aspects, said pharmaceutical composition comprises at least 5×10⁹ CFU to 10¹¹ CFU of said at least one probiotic microorganism. In some aspects, said pharmaceutical composition further comprises at least one VSC-releasing enzyme inhibitor, at least one sweetener, at least one filler, at least one prebiotic, at least one flavoring, at least one BLIS fermentation media remnant, or a combination thereof. In some aspects, said at least one VSC-releasing enzyme inhibitor comprises zinc. In some aspects, said at least one VSC-releasing enzyme inhibitor is zinc oxide. In some aspects, said at least one sweetener is a stevia leaf extract, xylitol, a derivative thereof, or a combination thereof. In some aspects, said at least one filler is microcrystalline cellulose, stearic acid, magnesium stearate, silicon dioxide, a derivative thereof, or a combination thereof. In some aspects, said at least one prebiotic is maltodextrin or a derivative thereof. In some aspects, said at least one flavoring is a natural mint flavor. In some aspects, said at least one BLIS fermentation media remnant is trehalose, lactitol, a derivative thereof, or a combination thereof. In some aspects, the number of beneficial bacteria is increased by at least two orders of magnitude. In some aspects, a number of detrimental bacteria is decreased by at least one order of magnitude. In some aspects, said pH is increased to at least about 7.25. In some aspects, said pH is increased to at least about 7.5. In some aspects, said one or more systemic disease pathways are inhibited by (i) out-crowding detrimental bacteria in biofilms that cover a surface of an oral cavity; (ii) producing bioactive compounds that target detrimental bacteria; (iii) stimulating the subject's defense mechanisms, or a combination thereof. In some aspects, the method further comprises formulating said pharmaceutical composition in an oral dosage form. In some aspects, said oral dosage form is a capsule or a tablet. In some aspects, said oral dosage form comprises a coating. In some aspects, said coating comprises at least one substance that enhances a taste, texture, nutritional value, efficacy, or a combination thereof of said oral dosage form. In some aspects, said coating comprises a prebiotic. In some aspects, said oral dosage form disintegrates in an oral cavity of a subject. In some aspects, said oral dosage form disintegrates in said oral cavity from 1 minute to 10 minutes after administration of said oral dosage form to said subject. In some aspects, said oral dosage form disintegrates in said oral cavity from 2 minutes to 5 minutes after administration of said oral dosage form to said subject. In some aspects, said subject is a human, a rodent, cat, a dog, a horse, a cow, or a bull. In some aspects, said subject is a human. In some aspects, said pharmaceutical composition is administered at least once daily. In some aspects, said pharmaceutical composition is administered at least twice daily. In some aspects, said pharmaceutical composition is administered at least three times daily.

In various aspects, provided herein is a method of treating or reducing the incidence of an oral condition, the method comprising orally administering to a subject in need thereof a pharmaceutical composition comprising a bacterial population comprising at least 4 bacterial species, wherein the bacterial population is present in an effective amount for treating or reducing the incidence of the oral condition in the subject. In some aspects, the oral condition is halitosis, a periodontal disease, or caries. In some aspects, the pharmaceutical composition is formulated in an oral dosage form. In some aspects, the oral dosage form is a tablet. In some aspects, the tablet disintegrates within 5 minutes after administration to the subject. In some aspects, the bacterial population inhibits the growth of at least one pathogenic microorganisms in the subject. In some aspects, the at least one pathogenic microorganisms is located in an oral cavity of the subject. In some aspects, the bacterial population provides a synergistic inhibitory effect against the at least one pathogenic microorganisms, compared to the bacterial species alone. In some aspects, the synergistic effect is determined by plating single bacterial species of the synergistic bacterial population in close proximity to a pathogenic microorganism onto a first agar plate, plating the synergistic bacterial population in close proximity to the pathogenic microorganism onto a second agar plate, and counting the number of pathogenic microorganism colonies on the first and second agar plate after an incubation time of at least 24 hours. In some aspects, the bacterial population comprises at least one Lactobacillus sp. In some aspects, the at least one Lactobacillus sp. is Lactobacillus reuteri, Lactobacillus salivarius, Lactobacillus brevis, Lactobacillus rhamnosus, Lactobacillus acidophilus, Lactobacillus plantarum, Lactobacillus casei, Lactobacillus paracasei, Lactobacillus curvatus, or a combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a boxplot graph showing the reduction of oral malodor in human subjects as measured by a Halimeter device in parts per million (ppm) of volatile sulphur compounds (VSC) over the course of two weeks. The subjects were treated with a pharmaceutical composition shown in TABLE 1 once daily for two weeks.

FIG. 2 illustrates a boxplot graph of saliva pH measurements in human subjects over the course of a two-week study. The subjects were treated with a pharmaceutical composition shown in TABLE 1 once daily for two weeks.

FIGS. 3A and 3B illustrate agar plates streaked with diluted saliva samples of two representative human study participants. The plates were grown under anaerobic conditions and were filled with an oral H2S organism with lead acetate (OHO—C) agar, which induces black precipitation in or around colonies that produce volatile sulphur compounds, indicating detrimental bacteria. The plates show a significant reduction in black colonies over the course of two weeks when the subjects were treated with a pharmaceutical composition shown in TABLE 1 once daily for two weeks. FIG. 3A illustrates agar plates streaked with diluted saliva samples of a study participant #1 taken at days 0, 7, and 14. FIG. 3B illustrates agar plates streaked with diluted saliva samples of a study participant #2 taken at days 0, 7, and 14.

FIG. 4 illustrates a graph depicting an observed change in the composition of the oral microbiome of a human subject at day 14 after a pharmaceutical composition comprising the ingredients shown in TABLE 1 was administered once daily for two weeks. The shift was measured through metagenomic DNA sequencing of the study participants' saliva samples pre- and post-treatment using the LEfSe (Linear Discriminant Analysis Effect Size) method, which determines the features (operational taxonomic units) most likely to explain differences between classes by coupling standard tests for statistical significance with additional tests encoding biological consistency and effect relevance.

FIG. 5A shows that the six different bacterial species, Lactobacillus brevis (#1), Lactobacillus salivarius (#2), Lactobacillus rhamnosus (#3), Lactobacillus acidophilus (#4), Lactobacillus plantarum (#5) and Lactobacillus reuteri (#6), were plotted in a pairwise all-against-all fashion on MRS plates in order to determine potential antagonizing effects among these different bacterial species.

FIG. 5B shows the observed inhibitory growth effects of L. rhamnosus on growth of L. reuteri (left set of images) and L. acidophilus (right set of images).

FIG. 6A shows a pairwise probiotic vs. pathogen bioassay layout to evaluate growth inhibition of probiotic species (shown in the “Probiotic Species” column of the table of FIG. 6A and labeled with letters A-F) against pathogens (shown in the “Pathogen Species” column of the table of FIG. 6A and labeled with numbers 1-9) that can inhabit the oral cavity of an animal.

FIG. 6B shows the results of the pairwise probiotic vs. pathogen efficacy bioassay shown in FIG. 6A over five replicates to treat or prevent gum health.

FIG. 7 shows the growth inhibitory effects of various probiotic consortia including Lactobacillus brevis, Lactobacillus salivarius, Lactobacillus rhamnosus, Lactobacillus acidophilus, Lactobacillus plantarum, and Lactobacillus reuteri against the pathogenic species Porphyromonas gingivalis (abbreviated and identified as “P.g.”). The bioassay data demonstrate hyper-additive effects (e.g., synergistic effects) when combining efficacious probiotics into a consortium of probiotics for the treatment of periodontal diseases.

FIG. 8A shows a pairwise probiotic vs. pathogen bioassay layout for tooth health experiments of probiotic species (shown in the “Probiotic Species” column of the table of FIG. 8A and labeled with letters A-J) against pathogens (shown in the “Pathogen Species” column of the table of FIG. 8A and labeled with numbers as shown) that can inhabit the oral cavity of an animal.

FIG. 8B shows the results of the pairwise probiotic vs. pathogen efficacy bioassay shown in FIG. 6A over two replicates to treat or prevent tooth health.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are compositions and methods for preventing, diagnosing, and/or treating a disease in a subject (e.g., a human). Such diseases can include oral disease such as halitosis, periodontal disease, caries, etc., as well as systemic conditions. Such systemic conditions may be caused by a dysbiosis in the subject inducing one or more of the following mechanisms or pathways that link oral infections to secondary systemic effects: (i) metastatic spread of an infection from an oral cavity as a result of transient bacteremia, (ii) metastatic injury from effects of circulating oral microbial toxins, and/or (iii) metastatic inflammation caused by immunological injury induced by oral microorganisms. The dysbiosis can be an oral dysbiosis and/or an intestinal dysbiosis.

In some instances, the disclosure provides methods and compositions for preventing and/or treating oral diseases. In such instances, compositions contemplated herein can comprise (i) at least one probiotic microorganism, (ii) at least one VSC-releasing enzyme inhibitor, and/or (iii) at least one sweetener; wherein the composition can comprise at least 10⁹ CFU of the at least one probiotic microorganism. Methods contemplated herein can comprise (i) administering a therapeutically effective amount of a pharmaceutical composition comprising at least one probiotic microorganism and (ii) increasing one or more of the following in an oral cavity of the subject: (a) a ratio of beneficial bacteria to detrimental bacteria to least about 1:1 and/or (b) a saliva pH to at least about 7.25. The methods provided herein to treat an oral disease or condition can comprise (i) administering a therapeutically effective amount of a pharmaceutical composition comprising at least one probiotic microorganism and (ii) increasing the number of beneficial bacteria in an oral cavity of the subject by at least one order of magnitude (10×) and/or reducing the number of pathogenic or detrimental bacteria by at least one order of magnitude (10×). Moreover, the compositions and methods described herein can be used to increase the pH in an oral cavity, e.g., the pH of saliva, into a neutral range. Such a neutral range can include pH levels from about 6.9 to about 7.5 in the oral cavity and/or the saliva of a subject.

Further provided herein are methods for preventing and/or treating a systemic condition in a subject. Such a systemic condition can be linked to, or has been caused by, an oral condition such as an oral dysbiosis in the subject. In such instances, the method for treating a systemic disease or conditions can comprise: (i) administering a therapeutically effective amount of a pharmaceutical composition comprising at least one probiotic microorganism and/or (ii) inhibiting one or more systemic disease pathways linked to the oral condition. In some instances, such systemic disease is a neurodegenerative disease such as Parkinson's disease or Alzheimer's disease.

The compositions and methods provided herein may offer unique advantages over conventional methods. Such advantages can include methods that are less disruptive and invasive and can be used to treat or reduce the incidence of a disease (e.g., an oral disease) in a subject. Such advantageous methods can comprise, e.g., (i) decreasing the number, concentration, and/or ratio, etc. of detrimental bacteria and/or (ii) increasing the number, concentration, and/or ratio, etc. of beneficial bacteria in an oral cavity of a subject. Such a decrease of detrimental or pathogenic bacteria and/or an increase of beneficial bacteria in a subject can be one or more orders of magnitude compared to an untreated subject. Moreover, the methods and compositions provided herein may offer long-term oral health (e.g., for several months and/or years) compared to conventional methods and compositions. Such long-term effects can be provided by, e.g., administering a pharmaceutical composition in certain time intervals to a subject. Such time intervals can include administering a pharmaceutical composition every day, every week, or every month, or a combination of various time intervals.

The unique and advantageous features of the methods and compositions provided herein were surprisingly found by combining certain microbial species and/or strains (e.g., Lactobacillus sp., Lactococcus sp., Bifidobacterium sp., Streptococcus sp., etc.) in specific combinations with one or more additional components (e.g., VSC-releasing enzyme inhibitors, sweeteners, prebiotics, etc.) in an oral formulation. When such compositions were administered to subject, it was found that such compositions can reverse an existing dysbiosis over time and restore a healthy microbiome (e.g., an oral and/or an intestinal microbiome) in a subject.

The term “subject,” as used herein, generally refers to an individual, such as a member of the animal kingdom. The subject may be living or not living. The subject can be a mammal. The subject can be a human. Non-limiting examples of other mammals can include rodents (e.g., mice and rats), primates (e.g., lemurs, monkeys, apes, etc.), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines (e.g., cows, bulls, etc.), donkeys, mules, bison, goats, camels, sheep, and deer). The subject can be a non-mammalian animal such as a turkey, a duck, or a chicken. The subject can be a farm animal (e.g., a pig, a goat, or a cow). The subject can be an aquatic animal (e.g., a fish or a crustacean).

The subject can be a living organism suffering from or prone to suffer from a disease or condition that can be diagnosed and/or treated using the methods and compositions provided herein. The subject may be a patient (e.g., a human patient) being treated or monitored by a healthcare provider (e.g., a primary care physician). Alternatively, the subject may not be a patient.

The term “isolated,” as used herein in the context of a microorganism (e.g., a bacterium, fungus, etc.), generally refers to a microorganism that has been (1) separated from at least some of the components with which it was associated when initially produced (whether in nature or in an experimental setting), and/or (2) produced, prepared, purified, and/or manufactured, e.g., using artificial culture conditions such as (but not limited to) culturing on a plate and/or in a fermenter. In some examples, isolated bacteria can include those bacteria that are cultured, even if such cultures are not monocultures. Isolated bacteria can be separated from at least about 10%, about 20%, about 30%, about 40%, about 50%, about 60%, about 70%, about 80%, about 90%, or more of the other components with which they were initially associated. Isolated bacteria can be more than about 80%, about 85%, about 90%, about 91%, about 92%, about 93%, about 94%, about 95%, about 96%, about 97%, about 98%, about 99%, or more than about 99% pure.

The terms “disease” and “condition,” as used herein, can be used interchangeably and generally refer to a state of being or health status of a patient or subject capable of being diagnosed and/or treated with a composition and method disclosed herein. The disease can be an inflammatory disease, an infectious disease, a (neuro-)degenerative disease, or an autoimmune disease. The disease can be associated with or suspected of being associated with one or more microorganisms. The disease can be associated with a microbiome of a subject, such as an oral, skin, vaginal, or gut (e.g., intestinal) microbiome. The disease can be a dysbiosis, such as an oral dysbiosis. The disease can be any condition associated with an oral microbial imbalance or dysbiosis, such as halitosis, periodontal disease, and caries.

The term “dysbiosis,” as used herein, generally refers to a difference in the oral and/or gastrointestinal microbiota compared to a healthy or general population. Dysbiosis can comprise a difference in oral and/or gastrointestinal microbiota commensal species diversity compared to a healthy or general population. Dysbiosis can also comprise a decrease of beneficial microorganisms and/or increase of pathobionts (pathogenic or potentially pathogenic microorganisms) and/or decrease of overall microbiota species diversity. Many factors can harm the beneficial members of the oral and/or intestinal microbiota leading to dysbiosis, including, but not limited to, antibiotic use, psychological and physical stress, radiation, and dietary changes. Dysbiosis can comprise or promote the overgrowth of a bacterial opportunistic pathogen. A dysbiosis can comprise a reduced amount (absolute number or proportion of the total microbial population) of bacterial and/or fungal cells of a species or genus, e.g., about 0%, 5%, 10%, 15%, 20%, 25%, 30%, 35%, 40%, 45%, 50%, 55%, 60%, 65%, 70%, 75%, 80%, 85%, 90%, 95%, or more lower compared to a healthy subject (e.g., a corresponding subject who does not have an oral conditions such as halitosis, periodontal disease, and/or caries, and who has not been administered an antibiotic within about 1, 2, 3, 4, 5, or 6 months, and/or compared to a healthy or general population). The dysbiosis can comprise an increased amount (absolute number or proportion of the total microbial population) of bacterial and/or fungal cells within a species or genus, e.g., about 5%, 10%, 15%, 20%, 25%, 30%, 35%, 40%, 45%, 50%, 55%, 60%, 65%, 70%, 75%, 80%, 85%, 90%, 95%, or more higher compared to a healthy subject (e.g., a corresponding subject who does not have an inflammatory disease, an infection, halitosis, periodontal disease, and/or caries, and who has not been administered an antibiotic within about 1, 2, 3, 4, 5, or 6 months, and/or compared to a healthy or general population). A subject who has or is suspected of having halitosis, periodontal disease, and/or caries, another oral and/or systemic condition (e.g., a gastrointestinal infection, gastrointestinal inflammation, or a neurodegenerative disease) or who has received an antibiotic within about 1, 2, 3, 4, 5, 6, 7, 8, 9, or 10 weeks can be deemed to comprise dysbiosis. An impaired microbiota can comprise small oral and/or intestinal bacterial or fungal overgrowth. Antibiotic administration (e.g., systemically, such as by intravenous injection or orally) can be a common and significant cause of major alterations in the normal microbiota. Other potential causes of dysbiosis can include changes in eating habits, medication, stress, or (increased) consumption recreational drugs, alcohol, smoking, etc. The methods and compositions provided herein can be used to diagnose, prevent, and/or treat a dysbiosis (e.g., oral dysbiosis) or a disease or condition (e.g., halitosis, periodontal disease, and/or caries) associated with dysbiosis in a subject.

The terms “treating” and “treatment,” as used herein in the context of a condition, disease, or disorder, or symptoms associated with a condition, disease, or disorder generally refer to an approach for obtaining beneficial results, including clinical results. Beneficial clinical results can include, but are not limited to, alleviation or amelioration of one or more symptoms or conditions; diminishment of extent of a condition, disorder, or disease; stabilization of the state of a condition, disorder, or disease; prevention of development of a condition, disorder, or disease; prevention of spread of a condition, disorder, or disease; delay or slowing of a condition, disorder, or disease progression; delay or slowing of a condition, disorder, or disease onset; amelioration or palliation of a condition, disorder, or disease state; and remission, whether partial or total. “Treatment” can also inhibit the progression of a condition, disorder, or disease; slow the progression of a condition, disorder, or disease temporarily; although in some instances, it can involve halting the progression of a condition, disorder, or disease permanently. For example, a method for treating a disease is considered to be a treatment if there is a 10% reduction in one or more symptoms of the disease in a subject as compared to a control. Thus, the reduction can be at least about 10%, 20%, 30%, 40%, 50%, 60%, 70%, 80%, 90%, 100%, or any percent reduction in between about 10% and about 100% as compared to native or control levels.

The term “diagnosis,” as used herein, generally refers to a relative probability that a disease (e.g., dysbiosis, halitosis, periodontal disease, caries, etc.) can be present in a subject (e.g., a human subject). Similarly, the term “prognosis” generally refers to a relative probability that a certain future outcome may occur in the subject with respect to a disease state. The methods and compositions provided herein can be used to diagnose a disease or condition such as a dysbiosis or an infection (e.g., the presence of one or more pathogenic microorganisms) in a subject.

The term “effective amount” generally refers to an amount necessary to produce a certain physiologic response (e.g., reduction of dysbiosis). Effective amounts and schedules for administering a therapeutic consortium may be determined empirically. The dosage ranges for administration can be large enough to produce the desired effect in which one or more symptoms of the disease or disorder are affected (e.g., reduced or delayed). The dosage may not be so large as to cause substantial adverse side effects, such as cross-reactions, anaphylactic reactions, and the like. Generally, the dosage may vary with the age, condition, sex, type of disease, the extent of the disease or disorder, route of administration, or whether other drugs are included in the regimen, and can be determined. The dosage can be adjusted in the event of any contraindications. Dosages can vary and can be administered in one or more dose administrations daily, for one or several days. For example, for the given parameter, an effective amount will show an increase or decrease of at least about 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least about a 1.2-fold, 1.5-fold, 2-fold, 5-fold, 10-fold, or more effect over a control. For prophylactic use, a therapeutically effective amount of the pharmaceutical compositions described herein are administered to a subject prior to or during early onset (e.g., upon initial signs and symptoms of an oral dysbiosis or a condition associated with an oral dysbiosis).

The abbreviation “sp.” for species, as used herein, generally refers to at least one species (e.g., 1, 2, 3, 4, 5, or more species) of the indicated genus. The abbreviation “spp.” for species, as used herein, generally refers to 2 or more species (e.g., 2, 3, 4, 5, 6, 7, 8, 9, 10, or more species) of the indicated genus. The methods and compositions provided herein can comprise a single species within an indicated genus or indicated genera, or 2 or more (e.g., a plurality comprising more than 2) species within an indicated genus or indicated genera.

The term “about,” as used herein in the context of a numerical value or range, generally refers to ±10% of the numerical value or range recited or claimed, unless otherwise specified.

Whenever the term “at least,” “greater than,” or “greater than or equal to” precedes the first numerical value in a series of two or more numerical values, the term “at least,” “greater than,” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Pharmaceutical Compositions

The present disclosure provides pharmaceutical compositions for restoring a healthy microbiome in a subject (e.g., a human). The microbiome can be an oral microbiome. The pharmaceutical compositions can restore a healthy oral microbial community by modifying (e.g., altering or changing) the microbial composition of the existing detrimental microbiome and, thereby, prevent and/or treat (e.g., cure) oral diseases such as halitosis, periodontal disease, and/or caries.

A pharmaceutical composition as provided herein can be designed and/or modulated to comprise one or more beneficial microorganisms (i.e., probiotic microorganisms) and one or more components and/or excipients in certain combinations and/or ratios. The amounts and/or types of probiotic species and the combination with additional component(s) and excipient(s) can give rise to the unexpected and unique properties of these compositions, including the ability to prevent and/or treat oral and/or systemic conditions. In some instances, such conditions can be treated in subjects with significantly different oral microbiome compositions and provide long-term oral health in these subjects.

In various instances, a pharmaceutical composition as provided herein can comprise one or more species and/or strains of probiotic bacteria that can be combined with one or more additional components and/or excipients including nutrients for the probiotics, VSC-releasing enzyme inhibitors, vitamins, minerals, sweeteners, flavorings, fillers, anti-caking agents, prebiotics, components of fermentation media, or a combination thereof.

### Probiotics

A pharmaceutical composition can comprise one or more genera, species, and/or strains of probiotic microorganisms (also referred to herein as “probiotics”). Such microorganisms can include bacteria, yeast, fungi, protozoa, etc. In some instances, a pharmaceutical composition can comprise one or more species and/or strains of bacteria. Such bacteria may be isolated bacteria. The one or more species and/or strains of bacteria can belong to one or more of the following phyla: Verrucomicrobia, Firmicutes, Proteobacteria, Actinobacteria, or Bacteroidetes, or a combination thereof.

A pharmaceutical composition as provided herein can comprise one or more species and/or strains of bacteria that may be of any one or more of the bacterial families of Coriobacteriaceae, Bacteroidaceae, Porphyromonadaceae, Bacillaceae, Paenibacillaceae, Lactobacillaceae, Clostridiaceae, Lachnospiraceae, Peptostreptococcaceae, Ruminococcaceae, Clostridiales, Erysipelotrichaceae, Acidaminococcaceae, Veillonellaceae, Enterobacteriaceae, Streptococcaceae, or Verrucomicrobiaceae, or a combination thereof.

A pharmaceutical composition as provided herein can comprise one or more species and/or strains of bacteria that may be of any one or more of the bacterial genera of Candida sp., Prevotella sp., Fusobacteria sp., Bacteroides sp., Bifidobacterium sp., Parabacteroides sp., Roseburia sp., Erysipelobacteriaceae sp., Enterobacteriaceae sp., Acidaminococcus sp., Faecalibacterium sp., Lachnospiracea sp., Enterobacteriaceae sp., Collinsella sp., Eubacterium sp., Lachnospiraceae sp., Lactococcus sp., Gammaproteobacteria sp., Dorea sp., Sporanaerobacter sp., Akkermansia sp., Bifidobacterium sp., Streptococcus sp., or Lactobacillus sp., or a combination thereof.

In some instances, a pharmaceutical composition of this disclosure can comprise one or more species and/or strains of Streptococcus sp. The Streptococcus sp. can be Streptococcus salivarius, Streptococcus acidominimus, Streptococcus agalactiae, Streptococcus alactolyticus, Streptococcus anginosus, Streptococcus saliviloxodontae, Streptococcus tangierensis, Streptococcus vestibularis, Streptococcus porcinus, Streptococcus cristatus, Streptococcus oralis ssp. dentisani (Streptococcus dentisani), Streptococcus gordonii, Streptococcus infantis, Streptococcus intermedius, Streptococcus oralis, Streptococcus rattus, Streptococcus sanguinis, Streptococcus thermophilus, Streptococcus uberis, or a combination thereof.

In some instances, a pharmaceutical composition of this disclosure can comprise one or more species and/or strains of Lactococcus sp. The Lactococcus sp. can be Lactobacillus bulgaricus, Lactobacillus casei, Lactobacillus casei subsp. casei, Lactobacillus delbrueckii subsp. lactis, Lactobacillus gasseri, Lactobacillus lactis subsp. cremoris, Lactobacillus lactis subsp. diacetylactis, Lactobacillus lactis subsp. lactis, Lactobacillus paracasei, Lactobacillus paracasei subsp. paracasei, Lactococcus lactis, Lactococcus chungangensis, Lactococcus formosensis, Lactococcus fujiensis, Lactococcus garvieae, Lactococcus plantarum, Lactococcus taiwanensis, or a combination thereof. In some instances, a pharmaceutical composition of this disclosure can comprise one or more species and/or strains of Lactococcus sp. Examples of Lactococcus sp. can include Lactococcus garvieae, Lactococcus lactis, or a combination thereof.

A pharmaceutical composition of this disclosure can comprise one or more species and/or strains of Lactobacillus sp. The Lactobacillus sp. can be Lactobacillus johnsonii, Lactobacillus rhamnosus, Lactobacillus zeae, Lactobacillus acidipiscis, Lactobacillus acidophilus, Lactobacillus agilis, Lactobacillus aviarius, Lactobacillus brevis, Lactobacillus coleohominis, Lactobacillus crispatus, Lactobacillus crustorum, Lactobacillus curvatus, Lactobacillus diolivorans, Lactobacillus farraginis, Lactobacillus fermentum, Lactobacillus fuchuensis, Lactobacillus harbinensis, Lactobacillus helveticus, Lactobacillus hilgardii, Lactobacillus intestinalis, Lactobacillus jensenii, Lactobacillus kefiranofaciens, Lactobacillus kefiri, Lactobacillus lindneri, Lactobacillus mali, Lactobacillus manihotivorans, Lactobacillus mucosae, Lactobacillus oeni, Lactobacillus oligofermentans, Lactobacillus panis, Lactobacillus pantheris, Lactobacillus parabrevis, Lactobacillus paracollinoides, Lactobacillus parakefiri, Lactobacillus paraplantarum, Lactobacillus plantarum, Lactobacillus pentosus, Lactobacillus pontis, Lactobacillus reuteri, Lactobacillus rossiae, Lactobacillus salivarius, Lactobacillus salivarius subsp. salivarius, Lactobacillus siliginis, Lactobacillus sucicola, Lactobacillus vaccinostercus, Lactobacillus vaginalis, Lactobacillus vini, or a combination thereof.

A pharmaceutical composition of this disclosure can comprise one or more species and/or strains of Bifidobacterium sp. The Bifidobacterium sp. can be Bifidobacterium angulatum, Bifidobacterium animalis, Bifidobacterium animalis subsp. lactis, Bifidobacterium asteroides, Bifidobacterium bifidum, Bifidobacterium boum, Bifidobacterium breve, Bifidobacterium catenulatum, Bifidobacterium choerinum, Bifidobacterium coryneforme, Bifidobacterium cuniculi, Bifidobacterium dentium, Bifidobacterium gallicum, Bifidobacterium gallinarum, Bifidobacterium indicum, Bifidobacterium longum, Bifidobacterium longum infantis, Bifidobacterium magnum, Bifidobacterium merycicum, Bifidobacterium minimum, Bifidobacterium pseudocatenulatum, Bifidobacterium pseudolongum, Bifidobacterium psychraerophilum, Bifidobacterium pullorum, Bifidobacterium ruminantium, Bifidobacterium saeculare, Bifidobacterium scardovii, Bifidobacterium simiae, Bifidobacterium stercoris, Bifidobacterium subtile, Bifidobacterium thermacidophilum, Bifidobacterium thermophilum, Bifidobacterium urinalis, or a combination thereof.

In some instances, a pharmaceutical composition herein can also comprise any one or more of Leptotrichia buccalis, Moraxella osloensis, Neisseria flava, Neisseria flavescens, Neisseria polysaccharea, Neisseria mucosa, Pediococcus acidilactici, Rothia mucilaginosa, Pediococcus pentosaceus, Selenomonas artemidis, Fusobacterium periodonticum, Fusobacterium naviforme (Fusobacterium nucleatum subsp. Vincentn), Gemella morbillorum, Kingella oralis, Weissella cibaria, or any combination thereof.

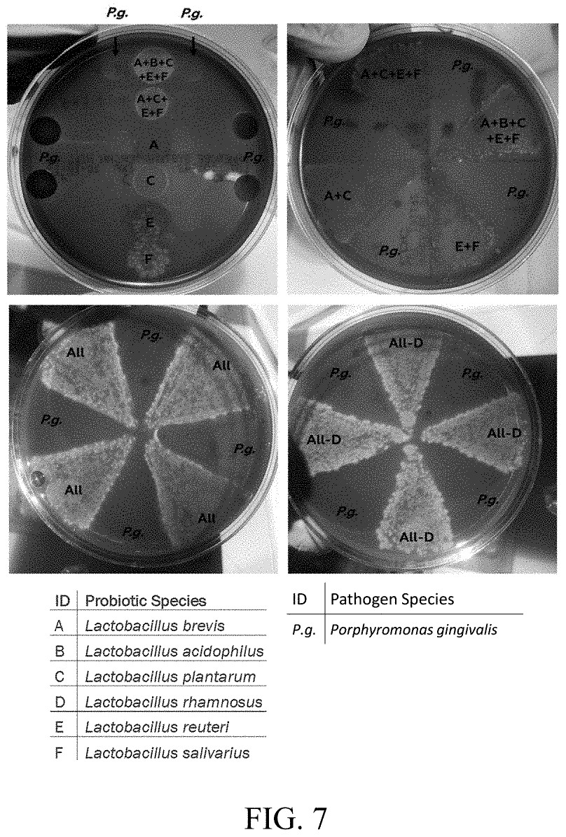

Thus, a pharmaceutical composition herein can comprise a probiotic bacterial species or a consortium of two or more probiotic bacterial species or strains. The two or more probiotics of such a consortium can elicit synergistic effects that can be beneficial for the prevention and/or treatment of a disease in a subject. Such synergistic effects of a bacterial consortium can be demonstrated by evaluating the ability of the consortium to inhibit the growth of a pathogenic bacteria, and then compare the measured inhibitory effect of the consortium with that of each bacterial species alone. In an example, a synergistic consortium of probiotic bacteria can inhibit the growth of pathogenic bacteria in, e.g., the oral cavity or the gut, of a subject as shown in FIG. 7 and described in EXAMPLE 4.

A pharmaceutical composition, e.g., one that is formulated in an oral dosage form (e.g., a capsule or a tablet), can comprise one or more species and/or strains of bacteria, either individually or in combination, at a concentration of at least about 10³, 10⁴, 10⁵, 10⁶, 10⁷, 10⁸, 10⁹, 10¹⁰, 10¹¹, 10¹², 10¹³, 10¹⁴, or more colony forming units per gram (CFU/g). The composition provided herein can be administered orally and include live microorganisms from about 10⁷ to about 10¹¹ CFU/g. The composition can include from about 10⁴ to about 10¹⁵ CFU/g of a bacterial species or strain. The composition can include from about 10⁵ to about 10¹⁵ CFU/g of a bacterial species or strain. The composition can include from about 10⁶ to about 10¹⁵ CFU/g of a bacterial species or strain. The composition can include from about 10⁷ to about 10¹⁵ CFU/g of a bacterial species or strain. The composition can include from about 10⁸ to about 10¹⁵ CFU/g of a bacterial species or strain. The composition can include from about 10⁹ to about 10¹⁵ CFU/g. The composition can include from about 10¹⁰ to about 10¹⁵ CFU/g of a bacterial species or strain. The composition can include from about 10¹¹ to about 10¹⁵ CFU/g of a bacterial species or strain. The composition can include from about 10¹² to about 10¹⁵ CFU/g of a bacterial species or strain. The composition can include from about 10¹³ to about 10¹⁵ CFU/g of a bacterial species or strain. The composition can include from about 10¹⁴ to about 10¹⁵ CFU/g of a bacterial species or strain. The composition can include from about 10⁸ to about 10¹⁰ CFU/g of a bacterial species or strain.

In various instances, a pharmaceutical composition as provided herein can comprise a therapeutically effective microbial consortium that comprises, consists essentially of, or consists of any 1, 2, 3, 4, 5, 6, 7, or 8, either per species/strain or combined, of Lactobacillus species and/or strains, Streptococcus species and/or strains, Lactococcus species and/or strains, and/or Bifidobacterium species and/or strains, or any combination thereof. Such a pharmaceutically effective pharmaceutical composition can comprise less than about 20, 19, 18, 17, 16, 15, 14, 13, 12, 11, 10, 9, 8, 7, 6, 5, 4, 3, or 2 different species and/or strains of bacteria.

Such bacterial species and/or strains can be used to modulate a composition of a subject's microbiome such as an oral microbiome. In some instances, the bacterial species and/or strains can populate an oral cavity of a subject and replace one or more detrimental bacterial species and/or strains that may be the cause of an oral condition, thereby restoring or substantially restoring a healthy microbiome in the subject.

### Nutrients and VSC-Releasing Enzyme Inhibitors

A pharmaceutical composition of this disclosure can comprise one or more compounds that may be used as nutrients and/or inhibitors of volatile sulfur compound (VSC)-releasing enzymes. Such compounds can include ammonium salts (e.g., cetylpyridinium chloride) and/or transition metal cations such as zinc, magnesium, silver, or copper, and any salts thereof. Transition metal salts that may be used in the pharmaceutical compositions described herein can include zinc chloride, zinc oxide, zinc acetate, copper chloride, copper acetate, or any combination thereof. A pharmaceutical composition that may be formulated in an oral dosage form (e.g., a capsule or a tablet) may comprise from about 0.5 milligram (mg) to about 15 mg, from about 1 mg to about 10 mg, from about 2 mg to about 8 mg, from about 4 mg to about 6 mg of nutrient and/or VSC-releasing enzyme inhibitors.

Such nutrients and/or VSC-releasing enzyme inhibitors can be used to reduce bad breadth in a subject. A pharmaceutical composition (e.g., a capsule or a tablet) can comprise zinc oxide as a nutrient and/or VSC-releasing enzyme inhibitor. The amount of zinc oxide in a pharmaceutical composition that can be formulated in an oral dosage form (e.g., a capsule or a tablet) can be from about 1 mg to about 10 mg, from about 2 mg to about 8 mg, or from about 4 mg to about 6 mg. The amount of zinc oxide in a pharmaceutical composition can be about 4 mg.

A pharmaceutical composition can further comprise nutrients such as vitamins and/or minerals. Vitamins can include vitamin C, a vitamin A, a vitamin D, a vitamin E, and/or one or more B vitamin(s). Such vitamins can support and enhance an immune system of a subject, e.g., a human. Minerals can include calcium, magnesium, sodium, iron, zinc, etc. Such minerals can be included in a pharmaceutical composition as salts, such as chloride salts, carbonate or bicarbonate salts, sulfate salts, etc.

### Sweeteners

A pharmaceutical composition of this disclosure can comprise one or more sweeteners and/or flavorings. Such sweeteners and/or flavorings can be non-cariogenic. Such sweeteners and flavorings can be plant extracts such as stevia leaf extracts; mint flavors; sugar alcohols such as erythritol, xylitol, sorbitol, maltitol, lactitol, isomalt, a derivative thereof, any combination thereof; or any other suitable sweeteners and/or flavorings. A flavoring, such as mint flavor, may be derived from an essential oil and thus may have antimicrobial properties itself. A pharmaceutical composition can comprise stevia leaf extracts, natural mint flavors, and xylitol in various ratios and amounts as sweeteners and/or flavorings.

A pharmaceutical composition that can be formulated in an oral dosage form (e.g., a capsule or a tablet) may comprise, either alone or in combination, from about 1 mg to about 1,200 mg, from about 5 mg to about 500 mg, from about 8 mg to about 250 mg, from about 10 mg to about 200 mg, from about 25 mg to about 150 mg, or from about 5 mg to about 100 mg of sweeteners and/or flavorings.

### Fillers

A pharmaceutical composition of this disclosure can comprise one or more fillers. Such fillers may be used to simplify measurement and/or preparation of an oral dosage form and/or to provide a homogenous weight and/or volume of an oral dosage form. Fillers can include microcrystalline cellulose, hydroxypropyl cellulose, stearic acid, magnesium stearate, silicon dioxide, lactose, sucrose, maltitol, sorbitol, xylitol, gelatin, a derivative thereof, or a combination thereof. A pharmaceutical composition of this disclosure can comprise microcrystalline cellulose, stearic acid, magnesium stearate, and silicon dioxide as fillers. A pharmaceutical composition that is formulated in an oral dosage form (e.g., a powder, a capsule, or a tablet) may comprise, either alone or in combination, from about 5% to about 80% of the one or more fillers by total formulation weight.

### Anti-Caking Agents

A pharmaceutical composition of this disclosure can comprise one or more anti-caking agents. An anti-caking agent may prevent the formation of lumps (e.g., caking) and may simplify packaging, transport, flowability, and/or administration of such oral dosage form by a subject. Anti-caking agents herein can include maltodextrin, (powdered) cellulose, magnesium stearate, stearic acid, silicon dioxide, a derivative thereof, or any combination thereof. A pharmaceutical composition of this disclosure can comprise maltodextrin as an anti-caking agent. A pharmaceutical composition that is formulated in an oral dosage form (e.g., a capsule or a tablet) may comprise, either alone or in combination, from about 0% to about 25% of the one or more anti-caking agents by total formulation weight.

### Prebiotics

A pharmaceutical composition of this disclosure can comprise one or more prebiotics. Prebiotics can include maltodextrin, other oligo and/or polysaccharides such as fructans and/or galactans, beta-glucans, xylooligosaccharides, or any combination thereof. A pharmaceutical composition of this disclosure can comprise prebiotics such as maltodextrin and related polysaccharides. A pharmaceutical composition that is formulated in an oral dosage form (e.g., a capsule or a tablet) may comprise, either alone or in combination, from about 0% to about 30% of the one or more prebiotics by total formulation weight The one or more prebiotics may be administered concurrently with the probiotic compositions, or prior to, or subsequent to administration of the probiotic composition in order to support growth and survival such probiotics. Thus, administration of prebiotics may enhance the efficacy of an administered (e.g., orally administered) composition described herein.

### Examples of Pharmaceutical Compositions

A pharmaceutical composition can comprise one or more species and/or strains of microorganisms (e.g., bacteria) and any one or more of sweeteners, flavorings, nutrients, VSC-releasing-enzyme inhibitors, fillers, prebiotics, anti-caking agents, and/or low amounts of fermentation media. Such fermentation media (e.g., BUS fermentation media), and components thereof (e.g., trehalose, lactitol, derivatives thereof, or other remnants), may be present in a composition in very low concentrations or trace amounts (e.g., in the parts per million (ppm) range or less).

A pharmaceutical composition as described herein may be formulated as a pharmaceutical composition suitable for administration to a subject (e.g., a human). Such pharmaceutical composition may be for oral administration (e.g., in a chewable tablet dosage form, a powder, a lozenge, a liquid, etc.). In some instances, the oral formulation is a tablet.

In some instances, a pharmaceutical composition as provided herein (or an oral formulation thereof, such as a tablet) may be used to prevent and/or treat halitosis. An example of such a composition can comprise one or more of the substances shown in TABLE 1. In some embodiments, such a pharmaceutical composition consists of, consists essentially of, or comprises the substances shown in TABLE 1.

TABLE 1
Example Composition for Treatment of Halitosis

| | Amount per tablet | | |
|---|---|---|---|
| | (overall tablet weight | | |
| Substance | of about 800 mg) | | Use |
| BLIS K12 Streptococcus | 20 | mg (2 Billion CFU) | Probiotic |
| salivarius | | | |
| BLIS M18 Streptococcus | 20 | mg (2 Billion CFU) | Probiotic |
| salivarius | | | |
| Lactococcus lactis | 3.34 | mg (2 Billion CFU) | Probiotic |
| Lactobacillus reuteri | 10 | mg (2 Billion CFU) | Probiotic |
| Lactobacillus salivarius | 50 | mg (2 Billion CFU) | Probiotic |
| Zinc (as zinc oxide) | 4 | mg | Nutrient and |
| | | | VSC-releasing |
| | | | enzyme inhibitor |
| Stevia leaf extract | 8.5 | mg | Non-cariogenic |
| | | | sweetener |
| Xylitol | 125 | mg | Non-cariogenic |
| | | | sweetener |
| Microcrystalline | 25-250 | mg | Filler |
| cellulose | | | |
| Stearic acid | 25-250 | mg | Filler |
| Magnesium stearate | 25-250 | mg | Filler |
| Maltodextrin | 25-250 | mg | Anti-caking |
| | | | agent and |
| | | | prebiotic |
| Silicon dioxide | 25-250 | mg | Filler |
| Natural mint flavor | 10-80 | mg | Flavor |

| | | |
|---|---|---|
| Trehalose | Traces | BLIS |
| | | fermentation |
| | | media remnant |
| Lactitol | Traces | BLIS |
| | | fermentation |
| | | media remnant |

In some instances, a pharmaceutical composition as provided herein (or an oral formulation thereof, such as a tablet) can be used to improve gum health and/or to treat diseases affecting gum health. An example of such a composition can comprise one or more of the substances shown in TABLE 2. In some embodiments, such a pharmaceutical composition consists of, consists essentially of, or comprises the substances shown in TABLE 2.

TABLE 2
Example Composition for Treatment of Gum Disease

| Substance | Amount per tablet | | Use |
|---|---|---|---|
| Lactobacillus brevis | 2 | Billion CFU | Probiotic |
| Lactobacillus salivarius | 2 | Billion CFU | Probiotic |
| Lactobacillus acidophilus | 2 | Billion CFU | Probiotic |
| Lactobacillus plantarum | 2 | Billion CFU | Probiotic |
| Lactobacillus reuteri | 2 | Billion CFU | Probiotic |
| Beta-glucans | 1-100 | mg | Prebiotic |
| Mannan Oligosaccharide | 1-100 | mg | Prebiotic |
| N-acetyl-D-mannosamine | 1-100 | mg | Prebiotic |
| Vitamin C | 100 | mg | Nutrient; antioxidant; |
| | | | immune enhancer; |
| | | | support of gum health |
| Vitamin D | 0.05 | mg | Nutrient; immune |
| | | | enhancer; support |
| | | | of gum health |
| Calcium | 100 | mg | Nutrient; immune |
| | | | enhancer; support |
| | | | of gum health |
| Iron | 15 | mg | Nutrient; immune |
| | | | enhancer; support |
| | | | of gum health |
| Zinc (as zinc oxide) | 4 | mg | Nutrient and |
| | | | VSC-releasing |
| | | | enzyme inhibitor |
| Stevia leaf extract | 8.5 | mg | Non-cariogenic |
| | | | sweetener |
| Xylitol | 125 | mg | Non-cariogenic |
| | | | sweetener |
| Microcrystalline cellulose | 25-250 | mg | Filler |
| Stearic acid | 25-250 | mg | Filler |
| Magnesium stearate | 25-250 | mg | Filler |
| Maltodextrin | 25-250 | mg | Anti-caking agent |
| | | | and prebiotic |
| Silicon dioxide | 25-250 | mg | Filler |
| Natural mint flavor | 10-80 | mg | Flavor |

In some instances, a pharmaceutical composition herein (or an oral formulation thereof, such as a tablet) can be used to improve tooth health and/or treat diseases affecting teeth health (e.g., tooth decay). An example of such composition can comprise one or more of the substances shown in TABLE 3. In some embodiments, such pharmaceutical composition consists of, consists essentially of, or comprises the substances shown in TABLE 3.

TABLE 3
Example Composition for Treatment of Tooth Decay

| Substance | Amount per tablet | | Use |
|---|---|---|---|
| Lactobacillus acidophilus | 2 | Billion CFU | Probiotic |
| Lactobacillus plantarum | 2 | Billion CFU | Probiotic |
| Lactobacillus salivarius | 2 | Billion CFU | Probiotic |
| Lactobacillus paracasei | 2 | Billion CFU | Probiotic |
| Lactobacillus case/ | 2 | Billion CFU | Probiotic |
| Streptococcus salivarius | 2 | Billion CFU | Probiotic |
| Beta-glucans | 1-100 | mg | Prebiotic |
| Mannan | 1-100 | mg | Prebiotic |
| Oligosaccharide | | | |
| N-acetyl-D-mannosamine | 1-100 | mg | Prebiotic |
| Vitamin A | 0.35 | mg | Tooth Health nutrient |
| Vitamin D | 0.05 | mg | Tooth Health nutrient |
| Calcium | 100 | mg | Tooth Health nutrient |
| Phosphorus | 200 | mg | Tooth Health nutrient |
| Zinc (as zinc oxide) | 4 | mg | Nutrient and |
| | | | VSC-releasing |
| | | | enzyme inhibitor |
| Stevia leaf extract | 8.5 | mg | Non-cariogenic |
| | | | sweetener |
| Xylitol | 125 | mg | Non-cariogenic |
| | | | sweetener |
| Microcrystalline | 25-250 | mg | Filler |
| cellulose | | | |
| Stearic acid | 25-250 | mg | Filler |
| Magnesium stearate | 25-250 | mg | Filler |
| Maltodextrin | 25-250 | mg | Anti-caking agent |
| | | | and prebiotic |
| Silicon dioxide | 25-250 | mg | Filler |
| Natural mint flavor | 10-80 | mg | Flavor |

In some instances, a pharmaceutical composition as provided herein (or an oral formulation thereof, such as a tablet) can be used to treat a systemic disease. Such a systemic disease can be a neurodegenerative disease such as Alzheimer's disease. An example of such a composition can comprise one or more of the substances shown in TABLE 4. In some embodiments, such a pharmaceutical composition consists of, consists essentially of, or comprises the substances shown in TABLE 4.

TABLE 4
Example Composition for Treatment of Systemic
Disease (here: Alzheimer's)

| Substance | Amount per tablet | | Use |
|---|---|---|---|
| Lactobacillus brevis | 3 | Billion CFU | Probiotic kills oral |
| | | | pathogens that may |
| | | | translocate to brain |
| Lactobacillus salivarius | 3 | Billion CFU | Probiotic kills oral |
| | | | pathogens that may |
| | | | translocate to brain |
| Lactobacillus plantarum | 3 | Billion CFU | Probiotic kills oral |
| | | | pathogens that may |
| | | | translocate to brain |
| Lactobacillus reuteri | 3 | Billion CFU | Probiotic kills oral |
| | | | pathogens that may |
| | | | translocate to brain |
| Niacin (Vitamin B3) | 8 | mg | Brain Health |
| Pyridoxine (Vitamin B6) | 20 | mg | Brain Health |
| Folate (Vitamin B9) | 800 | μg | Brain Health |
| Cobalamin (Vitamin B12) | 100 | μg | Brain Health |
| Ascorbic acid (Vitamin C) | 100 | mg | Brain Health |
| 25(OH)D (Vitamins | 25 | μg | Brain Health |
| D2 and D3) | | | |
| Tocopherols and | 2 | μg | Brain Health |
| tocotrienols (Vitamin E) | | | |
| Phylloquinone | 50 | μg | Brain Health |
| (Vitamin K1) | | | |
| (n-3) fatty acid | 200 | mg | Brain Health |
| docosahexaenoic acid | | | |
| (DHA) | | | |
| Zinc (as zinc oxide) | 4 | mg | Essential |
| | | | micronutrient |
| | | | and VSC release- |
| | | | enzyme inhibitor |
| Stevia leaf extract | 8.5 | mg | Non-cariogenic |
| | | | sweetener |
| Xylitol | 125 | mg | Non-cariogenic |
| | | | sweetener |
| Microcrystalline cellulose | 25-250 | mg | Filler |
| Stearic Acid | 25-250 | mg | Filler |
| Magnesium Stearate | 25-250 | mg | Filler |
| Maltodextrin | 25-250 | mg | Anti-caking agent |
| | | | and prebiotic |
| Silicon Dioxide | 25-250 | mg | Filler |
| Natural mint flavor | 10-80 | mg | Flavor |

### Orally Disintegrating Tablets

A pharmaceutical composition as provided herein may be formulated in an oral dosage form such as a powder, capsule or tablet. Such a powder, capsule or tablet may be produced and/or formulated such that it disintegrates in the mouth of a subject following oral administration. The disintegration rate of a powder, capsule or tablet comprising a therapeutic microbial consortium may be modulated. Modulation of a disintegration rate may be achieved, e.g., by using certain amounts and/or ratios of additional components (e.g., fillers, disintegrants, etc.) and/or by using certain production methods. In some instances, a certain disintegration rate can be achieved by using one or more disintegrants in a pharmaceutical composition. In an example, a tablet formulation comprising microcrystalline cellulose as a disintegrant can provide disintegration times of about 4-5 minutes.

Disintegration of a powder, capsule or tablet as described herein may provide distribution of the microbial composition and additional components (e.g., sweeteners, nutrients, etc.) in the oral cavity of a subject following administration. An increased and/or homogenous distribution of probiotics may provide increased therapeutic efficacy of the pharmaceutical compositions described herein. An increased disintegration rate of a powder, capsule or tablet in the oral cavity may minimize the overall disintegration time of the powder, capsule or tablet in the oral cavity which may provide a higher patient compliance. The disintegration time of a capsule or tablet in the oral cavity may be from about 10 seconds to about 10 minutes, from about 20 seconds to about 9 minutes, from about 30 seconds to about 8 minutes, from about 40 seconds to about 7 minutes, from about 50 seconds to about 6 minutes, from about 1 minute to about 5 minutes, or from about 2 minutes to about 4 minutes. The disintegration time of a powder, capsule or tablet in the oral cavity may be at most about 10 minutes, at most about 9 minutes, at most about 8 minutes, at most about 7 minutes, at most about 6 minutes, at most about 5 minutes, at most about 4 minutes, at most about 3 minutes, at most about 2 minutes, at most about 1 minute, at most about 45 seconds, or at most about 30 seconds.

In various instances, the disintegration time of an oral dosage form (e.g., powder, tablet or capsule) is less than about 8 minutes, less than about 5 minutes, or about 2-3 minutes.

### Production of Pharmaceutical Compositions

A pharmaceutical composition as described herein may be produced to comprise one or more viable and biologically active microorganisms. These microorganisms (e.g., bacteria) may be fermented individually and then lyophilized into a powder. Such lyophilized microorganisms may then be mixed with one or more other components of a pharmaceutical composition (e.g., fillers, prebiotics, nutrients, sweeteners, and/or flavorings). Prior to mixing, dispensation, transportation, and/or tablet pressing, the lyophilized microorganisms may be in a carrier matrix comprising, e.g., maltodextrin. Such a matrix may reduce caking and/or sheering of the lyophilized microorganisms during mixing, dispensing, transportation, and/or tablet pressing.

Following production, a pharmaceutical composition may be analyzed for its purity, composition, viability, and/or metabolic activity. Analysis may comprise use of bioinformatics to, e.g., determine the ratios of bacteria species and/or strains in a composition. In an example, raw probiotic powder can be tested for the formation of colony forming unit (CFU), a measure of live bacteria in the dried, raw material powder to ensure viability of the selected bacteria. To that end, a gram of powder can be re-hydrated with buffer, e.g., PBS and/or liquid growth medium (e.g., MRS broth), and a dilution series can be created from which plates can be inoculated. After 24-72 hours of growth at 37° C., the colonies on the plates can be counted and CFUs per gram can be calculated from the dilution factors of the plates. Consortia of bacteria can also be analyzed for purity and composition by DNA sequencing. For such analysis, a tablet from the final product can be dissolved and total DNA can be extracted with a commercial kit (e.g., MoBio Soil extraction kit). The purified DNA can then be prepared for amplicon-free sequencing according to the sequencing manufacturer's instructions (e.g., Illumina' instructions) and sequenced. This can be done using whole genome shotgun sequencing or 16S sequencing. Both methods can allow for the quantification of sequenced molecules, which can be traced back to the respective source organism. After normalization for several factors, this method can yield ratios for all strains in the original oral formulation (e.g., tablet or capsule). It can also allow the identification of any DNA that may not be part of the intended consortium and thus provides additional measurements to identify type and quantity of potential contaminants.

A pharmaceutical composition described herein may be produced in a way that provides high patient compliance. For example, a pharmaceutical composition may be produced such that is can be made available (e.g., commercially available) in containers such as stick packs, sachets, tablet bottles, spray bottles, etc. Such containers may provide pre-measure amounts of dosage forms, for example, that a subject may be able to administer (e.g., self-administer) in a specific dosage and/or time interval. The packaging may be designed to allow purchase of a monthly dose, and may further allow timely and convenient shipment, e.g., as a flat (e.g., <¾ inch) bubble-wrap envelope.

### Methods of Use

A pharmaceutical composition as provided herein can be used to prevent and/or treat a disease or condition in one or more subjects, even if multiple subjects have (e.g., have been diagnosed with) significantly different oral microbiome compositions and/or suffer from different oral diseases. Such broad efficacy coverage can be due to such a composition's modes of action.

A pharmaceutical composition can be designed and/or modulated to comprise one or more species or strains of microorganisms in combination with one or more additional components that can give rise to such modes of action. As an example, it has surprisingly been found that a pharmaceutical composition of the disclosure may be configured to provide overlapping modes of action from the different components of the formulation that may allow the pharmaceutical composition to be therapeutically effective in a diverse group of subjects with different oral microbiome compositions.

A pharmaceutical composition as disclosed herein may function via one or more of the following modes of action: (i) out crowding, (ii) production of natural bioactive compounds by such beneficial microorganisms, and/or (iii) stimulation of the host defense mechanisms. In out crowding, each dose of probiotics administered to and/or self-administered by a subject (e.g., a human) may contain billions of beneficial microorganisms that start to compete with the existing microbiome for nutrients and living space (e.g., adhesion to cell or tooth surface for long-term survival in the mouth). The existing microbiome may change, e.g., in its microbial compositions due to the supplemented microbial species administered. This effect by the probiotic composition may be amplified if the limited living space is cleaned off of the pre-existing microbes and biofilms (e.g. through tooth brushing, flossing, tongue scraping, etc.) and/or by supplying prebiotics, and/or by changes in the live style of a subject, e.g., by eating habits, hygiene habits, exercise regimen, medication/drug use, etc. In production of natural bioactive compounds by such beneficial microorganisms, bioactive compounds (e.g. bacteriocins, protein-derived toxins, etc.) may interrupt the growth of detrimental microbial species. Such an effect can range from slowing the growth rate of detrimental microbial species to actively killing the detrimental microbial species. Another effect may be the disruption of existing biofilms and/or disruption of new biofilm formation. In stimulation of the host defense mechanisms, a subject (e.g., a human) may have developed various defense mechanisms to limit the growth of detrimental microbes in the oral cavity. However, some detrimental bacteria may have evolved mechanisms and survival strategies to evade such host defense mechanisms. Hence, supplementing the existing oral microbial community with beneficial microorganisms (e.g., beneficial bacteria) may stimulate and/or enhance the existing host defense mechanisms (e.g., enhanced immune defense via symbiotic interactions) that may lead to a reduction in the amount and/or concentration of detrimental bacteria in the oral cavity.

A pharmaceutical composition can also be designed and/or modulated to have multi-tiered modes of action. Such multi-tiered modes of action can be provided by combining one or more microbial strains or species with certain amounts of and/or in certain ratios with additional components (e.g., nutrients, enzyme inhibitors, sweeteners, fillers, etc.). As an example, a pharmaceutical composition comprising a certain combination of peppermint, a zinc compound (e.g., zinc oxide), and one or more probiotic species (e.g., one or more Lactobacillus sp.) can surprisingly have the unique and complementary modes of action to (i) quickly (e.g., <1 hour after administration) mask bad breath in a subject using the peppermint; (ii) reduce bad breath over the following hours and/or days after administration using the zinc compound; and (iii) provide a long-term (e.g., several weeks to several months) reduction in bad breath using the probiotic species of such composition.

A pharmaceutical composition as described herein can be used to prevent and/or treat an oral disease or condition. Such an oral disease or condition may be associated with an oral dysbiosis. A pharmaceutical composition as described herein may be particularly useful for the prevention and/or treatment of oral diseases and conditions that may be associated with oral dysbiosis such as bad breath, inflammation of the gum, or tooth decay. The oral disease may be halitosis. The oral disease may be a periodontal disease. The oral disease may be caries.

Prevention and/or treatment of halitosis may result in fresh breath in a subject compared to a subject that did not receive such a composition. Prevention and/or treatment of a periodontal disease may improve gum health in a subject and/or protect a subject from diseases affecting gum health such as gingivitis (e.g., gum inflammation). Prevention and/or treatment of caries may improve tooth health in a subject and/or protect from diseases affecting tooth health (e.g., prevent/treat tooth decay).

A pharmaceutical composition (e.g., those comprising one or more nutrients and/or VSC-releasing enzyme inhibitors) can reduce VSCs in the oral cavity of a subject at least 2-fold, 3-fold, 4-fold, or more. Such nutrients and/or VSC-releasing enzyme inhibitors can reduce VSCs in the oral cavity of a subject to below 300 ppm, 200 ppm, 100 ppm, 50 ppm, or less as determined using saliva samples from the subject (see, e.g., EXAMPLE 2).

A pharmaceutical composition can modulate a pH in an oral cavity of a subject. The pH in the oral cavity (e.g., saliva pH) can be increased from an acidic pH (e.g., between pH 5.5 and pH 6.5) to a more neutral pH (e.g., between pH 6.5 and pH 7.5, or between pH 6.9 and pH 7.5). Such neutral pH can be provided through metabolic activity and an active pH change by metabolites secreted from the probiotics and/or due to beneficial bacterial eliminating detrimental microbes that may cause otherwise an acidic environment.

A pharmaceutical composition may be used to prevent and/or treat a systemic disease or condition. Such systemic disease or condition in a subject may be linked to and/or originate from an oral health condition such as oral conditions associated with oral dysbiosis. Such diseases that may be treated with the pharmaceutical compositions herein can include (but are not limited to) Alzheimer's disease, preterm birth, cardiovascular diseases (e.g., heart disease, stroke, etc.), arthritis, obesity, diabetes, bacterial pneumonia, and/or low-birth weight newborns.

The systemic disease may be Alzheimer's. Prevention and/or treatment of Alzheimer's may result in reduced severity of the disease in a subject compared to a subject that did not receive a composition as described herein.

The systemic disease may be preterm birth. Prevention of preterm birth may result in reduced severity of the disease in a subject compared to a subject that did not receive a composition as described herein. The systemic disease may be a low-birth weight in a newborn. Prevention of low-birth weight may result in reduced severity of the disease in a subject compared to a subject that did not receive a composition as described herein.

A systemic disease may be a cardiovascular disease. Prevention and/or treatment of cardiovascular diseases may result in reduced severity of the disease in a subject compared to subject that did not receive a composition as described herein.

A systemic disease may be obesity. The systemic disease may be diabetes mellitus. The systemic disease may be bacterial pneumonia.

A pharmaceutical composition as provided herein may prevent and/or treat systemic diseases via modification, suppression, and/or elimination of one or more of the following disease pathways: (i) metastatic infection, (ii) metastatic injury, and/or (iii) metastatic inflammation. With regard to metastatic injury, oral disease conditions and trauma in the oral cavity (e.g., after dental procedures) may cause transient bacteremia in which potentially pathogenic microorganisms breach the physical, electrical, and immunological barriers and enter the tissue and/or blood stream. Once circulating in the blood stream, these microorganisms may cause or contribute to disease development in other tissues and/or organs of a subject (e.g., in the brain, the heart, the lungs, etc.). With regard to metastatic injury, oral disease conditions may cause increased secretion of exotoxins and cytolytic proteins from microorganisms in the oral cavity that can diffuse into the bloodstream and cause systemic disease conditions throughout various tissues or organs of a subject (e.g., a human). With regard to metastatic inflammation, microorganisms involved in oral disease conditions may shed antigens that can enter the subject's blood stream and may cause the formation of immunocomplexes with matching antibodies. These complexes may trigger inflammatory reactions in various tissues or organs of a subject.

The compositions (e.g., oral probiotic compositions) described herein may modify, suppress, and/or eliminate systemic disease pathways linked to oral health by modification, reduction, and/or elimination of detrimental microorganisms, e.g., in the oral cavity of a subject. Thus, using the probiotic compositions of this disclosure (e.g., with or without additional substances such as prebiotics, vitamins, and other bioactive substance) may allow for (i) out crowding detrimental bacteria in the biofilms that cover the surfaces of the oral cavity; (ii) production of natural bioactive compounds that target (e.g., eliminate or reduce the presence of) detrimental microorganisms; and/or (iii) stimulation of the host (e.g., subject's) defense mechanisms to eliminate and/or reduce the presence of detrimental bacteria. The reduction of detrimental microorganisms (e.g., reduction of detrimental microorganism cell counts) in the oral cavity (e.g., at particular disease sites in the cavity) may reduce the incidence of metastatic pathways being triggered, thereby reducing and/or preventing the spreading of the disease to other tissues and/or organs (e.g., preventing systemic disease development or progression).

A pharmaceutical composition as provided herein can be used to reduce the abundance of one or more bacterial species or strains (e.g., detrimental bacterial species or strains). Such bacterial species or strains can belong to Plantomycetes, Sporichthyaceae, Planctomycetia, N1423WL, RB41, mb2424, Gemmatimonadetes, Bradyrhizobium, Acidimicrobiia, Acidobacteria_6, Haliangiaceae, Ellin6067, Ellin6075, Gemmatales, Gemmataceae, Gemmata, Segetibacter, Pirellulales, Gemm_1, Pirellulaceae, Ruminococcaceae, Selenomonas noxia, Schwartzia, Moraxellaceae, Prevotella, Prevotellaceae, Bacteroidia, or Bacteriodetes, or any combination thereof.

The pharmaceutical compositions described herein may be formulated for administration to a subject such as a human. The administration may be oral administration. Thus, the compositions disclosed herein may be formulated in oral dosage forms such as capsules of tablets (e.g., off-white chewable and/or disintegrating tablets). A tablet as described herein may comprise one or more coatings. A coating may be used to modulate a release rate of the probiotics and additional components from such oral dosage form. In some instances, a coating may provide sustained and/or controlled release of the components into the oral cavity. In some instances, a coating may provide accelerated release of the ingredients in the oral cavity. A coating of such a tablet or capsule may comprise one or more layers of prebiotics. A coating may comprise one or more substances that may enhance the taste, texture, nutritional value, and/or efficacy of the tablet. A coating may improve shelf life by reducing diffusion of oxygen and water vapor to the encapsulated probiotic formulation.

In some instance, a coating can be a film coating. Film coatings can contain at least one or more of polymer(s), plasticizer, colorant, opacifier, solvent, and/or carrier substance. The carrier substance may contain one or more flavor components (e.g., a natural mint flavor, orange flavor, or cherry flavor), one or more prebiotic components (e.g., mannan oligosaccharide, beta-glucans, or n-acetyl-d-mannosamine), one or more vitamin components (e.g., vitamin D, vitamin C, or vitamin B), one or more minerals (e.g. zinc, magnesium, or potassium), one or more plant components or extracts (e.g., ginger powder, parsley powder, or mint oil) and/or postbiotics, or a combination thereof.

A pharmaceutical composition as provided herein may be administered to a subject (e.g., a human subject) one or more times daily. In some instances, such compositions can be administered at least once daily, at least twice daily, at least three times daily, or more times daily. Two or more daily administrations may include administration at different times during the day, e.g., in the morning and/or in the evening during or after the regular oral hygiene routine. A pharmaceutical composition may be administered for at least 1, 2, 3, 4, 5, 6, 7, 8, 9, 10, 11, 12, 13, 14, 15, or more days. Such an administration may occur on at least 2, 3, 4, 5, 6, 7, 8, 9, 10, 11, 12, 13, 14, 15 or more consecutive days. A pharmaceutical composition may be administered for at least 1, 2, 3, 4, 5, 6, 7, 8, 9, 10, 11, 12, 13, 14, 15, or more weeks. A pharmaceutical composition may be administered for at least 1, 2, 3, 4, 5, 6, 7, 8, 9, 10, 11, 12, 13, 14, 15, or more months.

A pharmaceutical composition may induce a measurable (e.g., visually detectable and/or using an assay) therapeutic effect in a subject about 4 weeks, 3 weeks, 2, weeks, 1 week, 1 day, or earlier after administration (e.g., oral administration). In some instances, a therapeutic effect can be detected at most about 2 weeks after administration, or earlier. In some instances, a therapeutic effect can be detected at most about 1 week after administration, or earlier.

A pharmaceutical composition as described herein may be used in animals, for example, pets that may live with humans and where bad breath or other oral diseases may be prevented and/or treated (e.g., in dogs, cats, etc.), or in larger, more resource intensive animals that may have longer life spans in which oral and/or dental treatments and prevention measures may be applied (e.g., in horses, cows, breeding bulls, other farm animals, etc.).

### EXAMPLES

The following examples merely illustrate the disclosure, and are not intended to limit the disclosure in any way.

### Example 1

### Production and Quality Control of an Oral Probiotic Composition

This example demonstrates the production and quality control of the oral probiotic composition shown in TABLE 2 that may be used to prevent and/or treat an oral disease or condition associated with oral dysbiosis (e.g., a gum disease).

The bacterial species Lactobacillus brevis, Lactobacillus salivarius, Lactobacillus acidophilus, Lactobacillus plantarum, and Lactobacillus reuteri were fermented individually and then lyophilized into a powder. The lyophilized microorganisms were then mixed with zinc oxide, stevia leaf extract, xylitol, microcrystalline cellulose, stearic acid, magnesium stearate, maltodextrin, silicon dioxide, and natural mint flavor. The composition was then produced into a tablet formulation.

Following production, such a tablet formulation may be analyzed for its purity, composition, viability, and/or metabolic activity. Analysis may comprise use of bioinformatics to, e.g., determine the ratios of bacteria species and/or strains in such composition prior to administration.

### Example 2

### Study Assessing the Improvement of Oral Malodor (Halitosis) in Human Subjects Using an Oral Microbial Composition

For each target condition (i.e., halitosis, periodontal disease, and caries) bacterial species were selected to treat the target condition. Experiments were conducted using combinations of two or more of the selected bacterial species and it was demonstrated that the selected species work synergistically together (see, e.g., EXAMPLE 5 below). Another set of experiments yielded the optimal composition and ratios of these species in terms of CFUs, concentrations of probiotics, etc. The most promising formulation for each condition was tested in a small-scale product study with human volunteers. For halitosis, the study demonstrated significant drops in oral malodor in at least 75% of cases after two weeks of a once daily application of the orally administrable composition of TABLE 1. TABLE 5 below shows an example setup of the study.

TABLE 5
Example Study Setup

| | |
|---|---|
| Purpose: | Study the beneficial influence of a composition |
| | comprising the ingredients shown in |
| | TABLE 1 on subjects suffering from halitosis |
| Number of | 18 |
| participants: | |
| Study duration: | 2 weeks (continuous enrollment) |
| Overall study | 1 Month |
| timeframe: | |
| Inclusion | Halimeter readings of >=200 ppm (noticeable |
| criteria: | bad breath) |
| | History of friends and family commenting |
| | negatively about breath quality |
| Exclusion | Immunocompromised, pregnant |
| criteria: | or breast feeding |
| | Allergic against any ingredient |
| | of the probiotic |
| | Children |
| Sampling | Day 0: before any composition described |
| | herein has been taken (control) |
| timepoints: | Day 7: after one week of taking a composition |
| | comprising the ingredients shown in |
| | TABLE 1 |
| | Day 14: after two weeks taking a composition |
| | comprising the ingredients shown in |
| | TABLE 1 |
| Collected data | 3 Halimeter readings (e.g., for direct |
| on each visit: | measurement of breath quality) |
| | 1 saliva pH measurement (e.g., as an |
| | indicator of oral health) |
| | 1 tongue swab for microbial plating on |
| | OHO-C agar for quantification of |
| | VSC-producing colonies |
| | 2 tongue swabs for microbiome sequencing |
| | (e.g., to determine exact shifts in microbiome |
| | composition) |
| | 1 picture of the tongue (e.g., for assessment of |
| | tongue coating) |
| | Qualitative feedback from participants |
| Study | Take one tablet each evening after completion |
| instruction: | of regular oral hygiene routine and before |
| | sleeping. |
| | Place tablet on tongue and let it dissolve while |
| | spreading saliva throughout the mouth. |
| | Avoid eating and drinking until the next |
| | morning. |
| Sampling day | Do not smoke, eat, or drink in the morning |
| instructions | before visit is completed, except |
| (on day 0, 7, | where medically necessary. |
| and 14): | |

### Study Results

### Oral Malodor Reduction

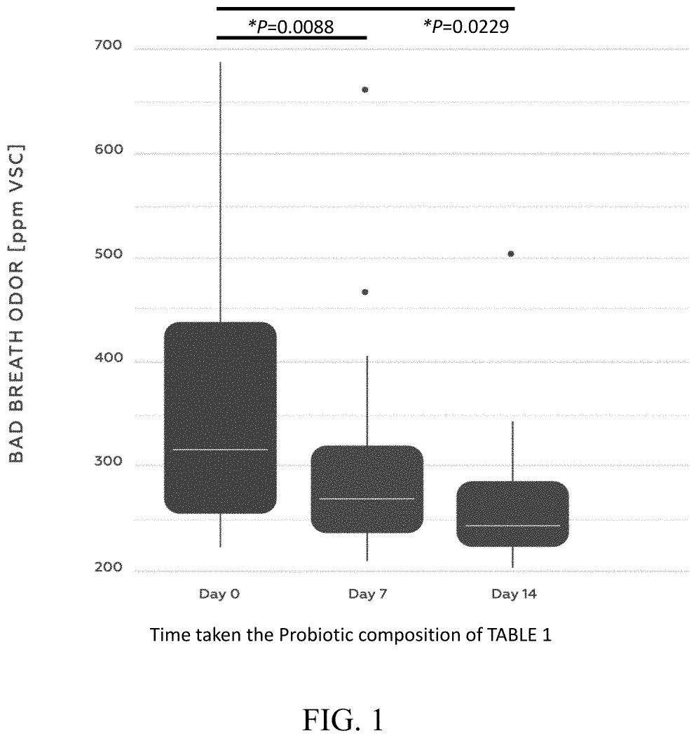

The daily intake of one tablet comprising the ingredients shown in TABLE 1 significantly reduced the oral malodor readings of the volunteers (FIG. 1) over the course of only two weeks. This was measured using a Halimeter device, which detects certain volatile sulphur compounds (VSC), one of the primary offending chemicals in oral malodor.

### Establishment of Healthy Saliva pH

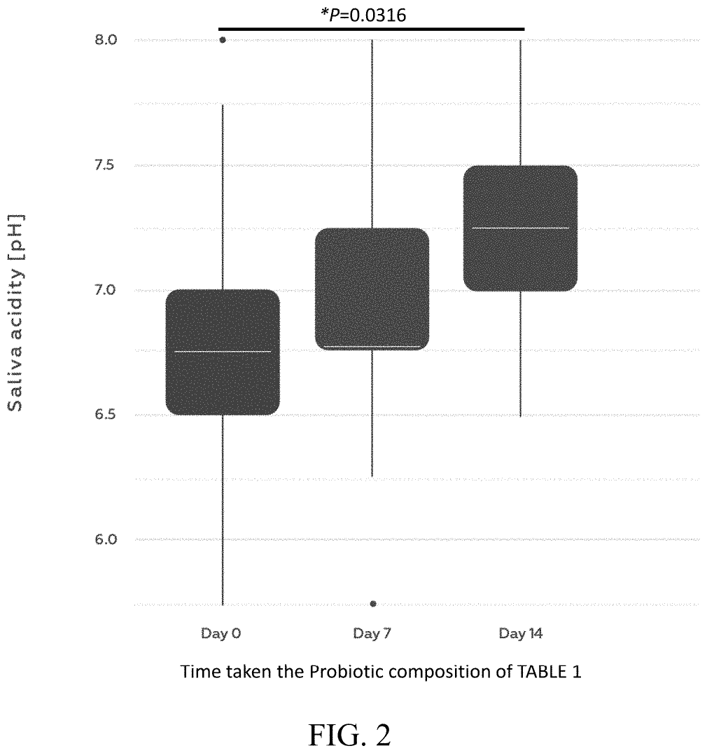

Acidic saliva levels were often associated with oral health conditions. During the study, the volunteers' saliva pH was measured with pH strips. As shown in FIG. 2, it was found that the average saliva pH increased significantly from an acidic to a neutral level, indicating a positive change in the oral microbiome from a diseased to a healthy state.

### Reduction of Bacteria Producing Volatile Sulphur Compounds (VSCs)

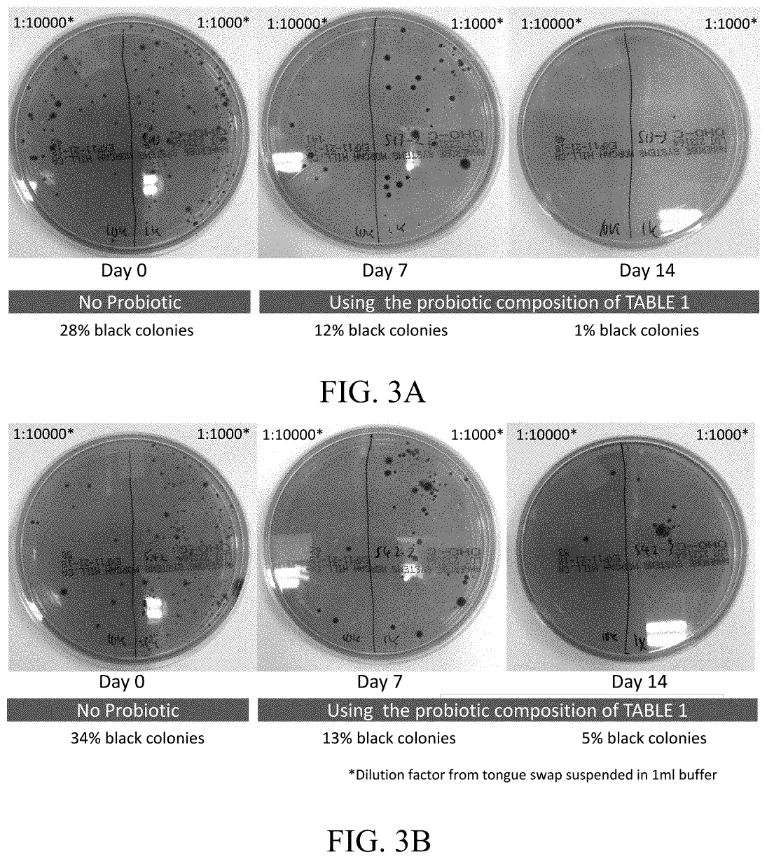

Diluted saliva samples were grown on oral H2S organisms agar with lead acetate (OHO—C) in agar plates under anaerobic conditions. Under these conditions, bacterial colonies that produce volatile sulphur compounds (in particular hydrogen sulphide) form black colonies due to the lead acetate reacting with the hydrogen sulfide, which causes a black precipitate to form within the colonies or on the agar surrounding them. In support of the previous two experimental setups, this experiment also confirmed the reduction of detrimental bacteria in the volunteers' oral microbiomes. FIG. 3A and FIG. 3B show the reduction of black colonies for two representative volunteers over the course of two weeks.

### The Oral Microbiome is Shifting to a Healthy State

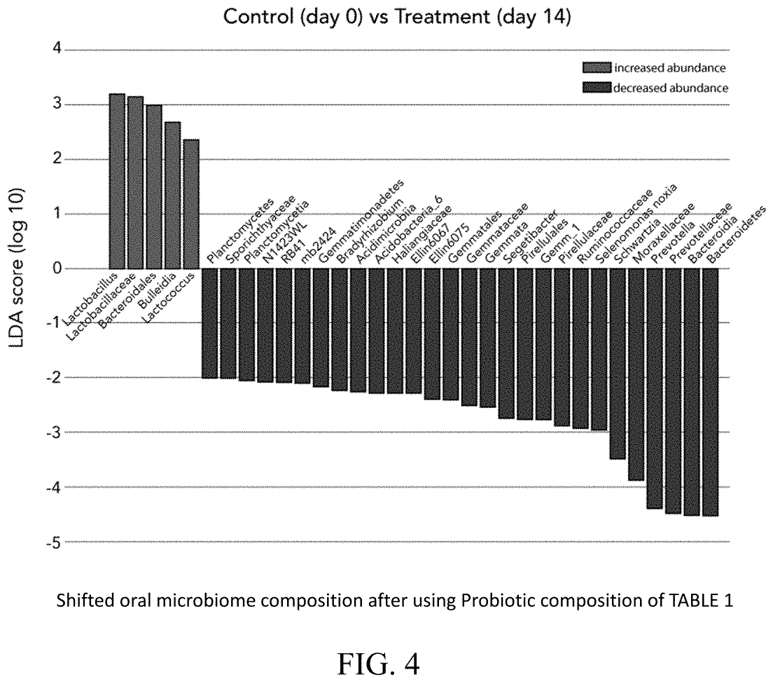

Metagenomic 16S DNA samples were taken from all participants during each visit. Bioinformatic and statistical analyses demonstrated a shift in the oral microbiomes of the volunteers after starting to take the orally administered composition. This data demonstrates that the oral probiotic compositions of this disclosure can result in a reduction of detrimental bacteria, such as Prevotella species, in the oral cavity while increasing the presence of beneficial bacteria such as Lactobacillus sp. and Lactococcus sp. As an example, FIG. 4 shows a list of differentially detected operational taxonomic units after two weeks of administration of a probiotic composition of the present disclosure. The shift in the microbial composition of the oral microbiome was measured through metagenomic DNA sequencing of the study participants' saliva samples pre and post treatment using the LEfSe (Linear Discriminant Analysis Effect Size) method.

### Example 3

### Study Assessing the Compatibility of Probiotic Oral Microbial Species for Formulation Optimization

This example describes a study assessing the compatibility of various bacterial species for formulation optimization.

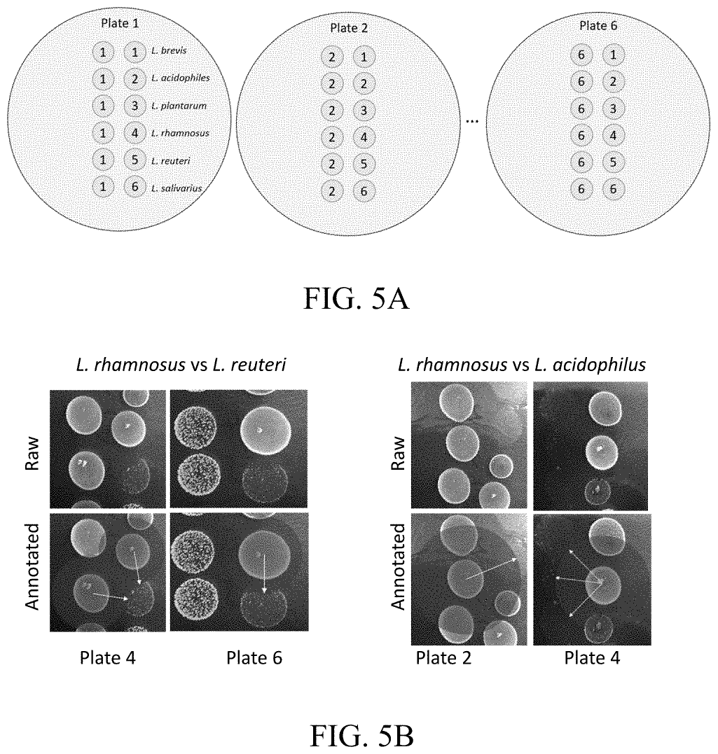

The probiotic bacterial species Lactobacillus brevis (#1), Lactobacillus salivarius (#2), Lactobacillus rhamnosus (#3), Lactobacillus acidophilus (#4), Lactobacillus plantarum (#5) and Lactobacillus reuteri (#6) were individually grown in MRS broth (Alpha Biosciences, Baltimore, Md., USA) for 48 hours at 37° C. Subsequently, 10 μl aliquots of each species in a broth solution was plotted in a pairwise all-against-all fashion on MRS plates as shown in FIG. 5A. The plates were then incubated aerobically for 48 hours at 37° C.

The results as shown in FIG. 5B indicate that L. rhamnosus exhibited inhibitory effects against L. reuteri and L. acidophilus. Thus, in some embodiments, formulations as provided herein comprising a combination of bacterial species can be optimized to not include L. rhamnosus formulations. All other bacterial species that were analyzed in this study did not show antagonizing effects and were deemed compatible for oral formulations described herein.

### Example 4

### Study Assessing the Efficacy of Probiotic Oral Microbial Species Against Pathogens Causing Periodontal Disease in Humans Through In-Vitro Bioassays

This example demonstrates the inhibitory efficacy of probiotic species against various oral pathogens implicated in the onset and progression of periodontal diseases.

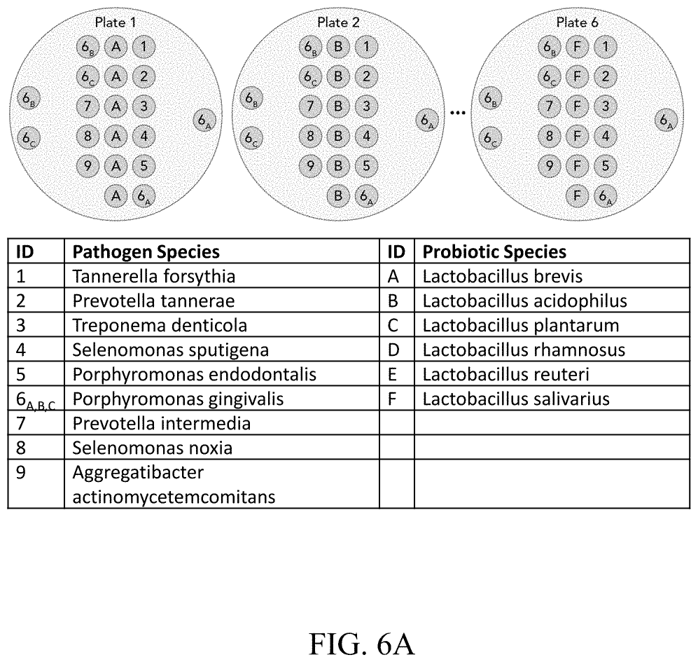

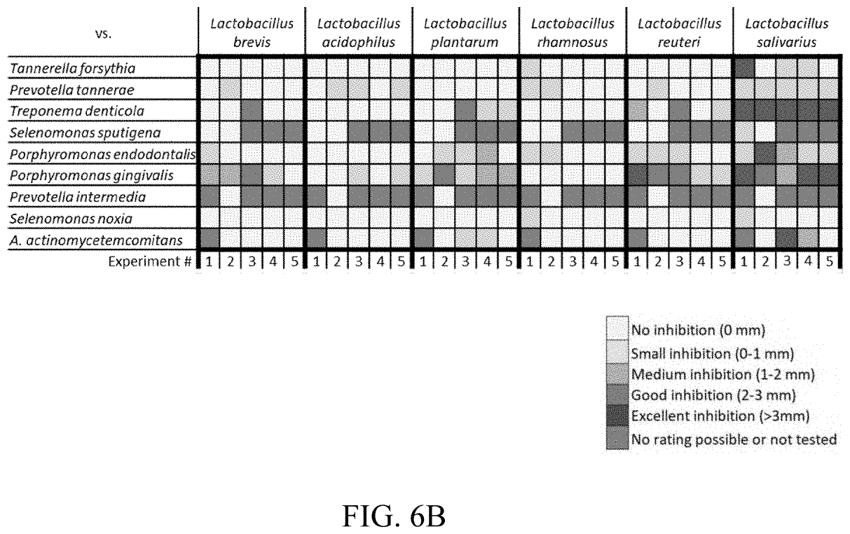

The probiotic bacterial species Lactobacillus brevis, Lactobacillus salivarius, Lactobacillus rhamnosus, Lactobacillus acidophilus, Lactobacillus plantarum and Lactobacillus reuteri were individually grown in MRS broth (Alpha Biosciences, Baltimore, Md., USA) for 48 hours at 37° C. Additionally, the oral pathogens Tannerella forsythia, Prevotella tannerae, Treponema denticola, Selenomonas sputigena, Porphyromonas endodontalis, Porphyromonas gingivalis, Prevotella intermedia, Selenomonas noxia, and Aggregatibacter actinomycetemcomitans were individually grown under anaerobic condition in THIO broth (Anaerobe Systems, Morgan Hill, Calif., USA), CRM broth (BD 218081) and/or CDC plates (Anaerobe Systems, Morgan Hill, Calif., USA), respectively. Subsequently, 10 μl aliquots of each probiotic was plotted on CDC plates and incubated for 48 hours. Pathogens were then plotted in close proximity to the probiotics as depicted in FIG. 6A. The plates were then further incubated anaerobically for 72 hours at 37° C. before visual assessment commenced. This experiment was repeated five times. A summary of the results is shown in FIG. 6B.

Consistent strong inhibition of pathogens was observed primarily by L. brevis, L. salivarius, L. reuteri and L. plantarum particularly against P. gingivalis, P. endodontalis and T denticola. L. salivarius further inhibited A. actinomycetemcomitans, T. forsythia and P. tannerae. The experiment was also repeated with OD 0.1 normalized cultures of probiotics and pathogens. The results of the normalization experiment did not differ from the non-normalized experiments.

### Example 5

### Study Assessing the Efficacy of Probiotic Oral Microbial Compositions Against Pathogens Causing Periodontal Disease in Humans Through In-Vitro Bioassays

This example demonstrates the inhibitory efficacy of consortia of probiotics against pathogens, in particular Porphyromonas gingivalis.

The probiotic bacterial species Lactobacillus brevis, Lactobacillus salivarius, Lactobacillus rhamnosus, Lactobacillus acidophilus, Lactobacillus plantarum and Lactobacillus reuteri were individually grown in MRS broth (Alpha Biosciences, Baltimore, Md., USA) for 48 hours at 37° C. Subsequently, equal amounts of each probiotic were mixed in different combinations and 10 μl of each mixture was plotted on CDC (tryptic soy agar) plates (Anaerobe Systems, Morgan Hill, Calif., USA). Similarly, 25 μl of each mixture was used to inoculate a slice of CDC plates (see FIG. 7). After 48 hours in 37° C., the plates were transferred to an anaerobic environment and further inoculated with P. gingivalis in 10 μl plots or 25 μl on slice-layout plates. Anaerobic incubation for 73 hours was performed before the plates were analyzed as shown in FIG. 7.

In all cases, a strong synergistic effect between bacterial species was observed that appeared to be stronger than the additive effect of the individual probiotics. The inhibitory effect was sufficient to completely inhibit the growth zone of P. gingivalis. In the dot-plot layout, all close-proximity plots were completely inhibited whereas the remote plots (acting as controls) grew largely unaffected. Since P. gingivalis is a key pathogen in the onset and progression of periodontal diseases, these findings suggest that the identified probiotic consortia described herein, particularly including the species L. brevis, L. reuteri, L. salivarius and L. plantarum in particular, may be, either alone or in combination, be suitable for effective treatment of periodontal disease through elimination of P. gingivalis and other periodontal pathogens in the oral cavity. Thus, these bioassay data demonstrate hyper-additive effects when combining efficacious probiotics into a consortium of probiotics for the treatment of periodontal diseases.

Moreover, and without being bound by any theory, it is assumed that, by inhibiting growth and/or eliminating P. gingivalis in the oral cavity, the probiotic consortia as described herein may be used to prevent such pathogens from entering the blood stream and ultimately preventing Alzheimer's and other systemic diseases.

### Example 6

### Study Assessing the Efficacy of Probiotic Oral Microbial Species Against Pathogens Causing Tooth Decay in Humans Through In-Vitro Bioassays

This example demonstrates the inhibitory efficacy of probiotic species against various oral pathogens implicated in the onset and progression of tooth decay.

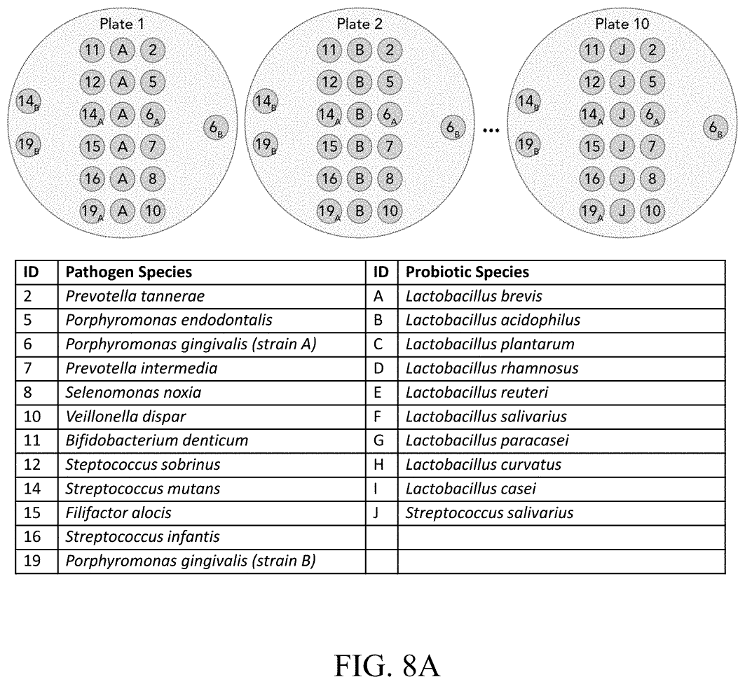

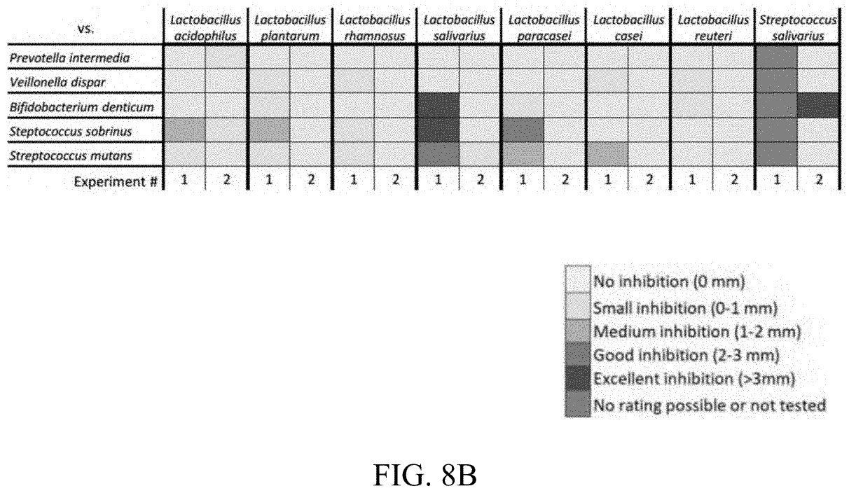

To that end, the probiotic bacterial species Lactobacillus acidophilus, Lactobacillus plantarum, Lactobacillus rhamnosus, Lactobacillus salivarius, Lactobacillus paracasei, Lactobacillus casei, and Lactobacillus reuteri were individually grown in MRS broth (Alpha Biosciences, Baltimore, Md., USA) for 48 hours at 37° C. Streptococcus salivarius powder was resuspended in PBS buffer prior to plating. Likewise, the oral pathogens Streptococcus mutans, Bifidobacterium denticum, Veillonella dispar, Prevotella intermedia, and Steptococcus sobrinus were individually grown under anaerobic conditions in THIO broth (Anaerobe Systems, Morgan Hill, Calif., USA), CRM broth (BD 218081), and/or CDC plates (Anaerobe Systems, Morgan Hill, Calif., USA), respectively. Subsequently, 10 μl of each probiotic broth solution was plotted on CDC plates and incubated for 48 hours. Pathogens were then plotted in close proximity to the probiotics as depicted in FIG. 8A. The plates were then further incubated anaerobically for 72 hours at 37° C. before visual assessment commenced. This experiment was repeated two times.

A summary of the results is shown in FIG. 8B. Strong growth inhibition of pathogens was observed by L. salivarius and S. salivarius against Bifidobacterium denticum. L. salivarius, L. plantarum, L. acidophilus, and L. paracasei showed significant growth inhibition of Streptococcus sobrinus. L. salivarius, L. paracasei, and L. casei showed significant growth inhibition against S. mutans. Thus, these data demonstrate that selected probiotics as described herein can be used alone, as a consortium of two or more probiotics, and/or in combination with one or more additional substances (e.g., prebiotics, nutrients, fillers, etc.) to treat and/or prevent various oral as well as systemic diseases.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein, which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-80. (canceled)

81. A composition comprising:
(i) at least one probiotic microorganism;
(ii) at least one volatile sulfur compound (VSC)-releasing enzyme inhibitor; and
(iii) at least one sweetener.

82. The composition of claim 81, wherein said at least one probiotic microorganism comprises at least one Lactobacillus sp., at least one Lactococcus sp., at least one Bifidobacterium sp., at least one Streptococcus sp., or a combination thereof.

83. The composition of claim 82, wherein said at least one Lactobacillus sp. is Lactobacillus reuteri, Lactobacillus salivarius, Lactobacillus brevis, Lactobacillus rhamnosus, Lactobacillus acidophilus, Lactobacillus plantarum, Lactobacillus casei, Lactobacillus paracasei, Lactobacillus curvatus, or a combination thereof.

84. The composition of claim 82, wherein said at least one Lactococcus sp. is Lactococcus lactis.

85. The composition of claim 82, wherein said at least one Bifidobacterium sp. is Bifidobacterium longum.

86. The composition of claim 82, wherein said at least one Streptococcus sp. is Streptococcus salivarius.

87. The composition of claim 86, wherein said Streptococcus salivarius strain comprises Streptococcus salivarius K12, Streptococcus salivarius M18, or a combination thereof.

88. The composition of claim 81, wherein said at least one probiotic microorganism is present in an amount from 10⁹ CFU to 10¹¹ CFU.

89. The composition of claim 81, wherein said at least one VSC-releasing enzyme inhibitor comprises a transition metal or a salt thereof.

90. The composition of claim 89, wherein said transition metal is zinc, magnesium, silver, or copper.

91. The composition of claim 89, wherein said at least one VSC-releasing enzyme inhibitor comprises zinc oxide.

92. The composition of claim 81, wherein said at least one sweetener is a stevia leaf extract, xylitol, a derivative thereof, or a combination thereof.

93. The composition of claim 81, further comprising at least one filler.

94. The composition of claim 81, further comprising at least one prebiotic.

95. The composition of claim 81, further comprising any one or more of a flavoring, a vitamin, a fermentation media remnant, or a mineral.

96. The composition of claim 81, formulated in an oral dosage form.

97. The composition of claim 96, wherein said oral dosage form is a capsule, a powder, or a tablet.

98. The composition of claim 97, wherein said oral dosage form is configured to disintegrate in an oral cavity of a subject.

99. A method for treating an oral condition in a subject in need thereof, said method comprising:
(i) administering an effective amount of a composition comprising at least one probiotic microorganism to the subject;
(ii) changing one or more of the following in an oral cavity of the subject compared to an untreated subject:
(a) increasing a number of beneficial bacteria by at least one order of magnitude; and/or
(b) normalizing the saliva pH to a range between 6.2-7.4; and
(iii) decreasing a number of pathogenic bacteria by at least one order of magnitude in the oral cavity of the subject compared to an untreated subject.

100. A method for treating a systemic condition in a subject that is linked to or has been caused by an oral condition in said subject, said method comprising:
(i) administering an effective amount of a composition comprising at least one probiotic microorganism; and
(ii) inhibiting one or more systemic disease pathways linked to said oral condition.
